(19) United States
(12) Patent Application Publication
(10) Pub. No.: US 2004/0002441 A1
(43) Pub. Date: Jan. 1, 2004

(54) Recombination modulators and methods for producing and using the same

(76) Inventors: Anca Segall, San Diego, CA (US); Clemencia Pinilla, Cardiff, CA (US)

Correspondence Address: PENNIE AND EDMONDS, 1155 AVENUE OF THE AMERICAS, NEW YORK, NY, 100362711

(21) Appl. No.: 10/418,943

(22) Filed: Apr. 17, 2003

Related U.S. Application Data
(62) Division of application No. 09/602,087, filed on Jun. 22, 2000, now abandoned.

(51) Int. Cl. C12Q 1/68; A61K 38/17; A61K 31/4745

(52) U.S. Cl. 514/2; 435/6; 514/283

(57) ABSTRACT

The present invention generally relates to cell growth modulators, methods of screening for such modulators and methods of using such modulators. In particular, the present invention provides a method of identifying a modulator of cell growth, which method comprises: a) assessing activity of a site-specific DNA recombinase or a type I DNA topoisomerase in the presence of a test substance; b) assessing activity of said site-specific DNA recombinase or said type I DNA topoisomerase in the absence of said test substance; and c) comparing said activities assessed in steps a) and b), whereby a difference in said activity assessed in step a) and said activity assessed in step b) indicates that said test substance modulates cell growth. Peptide cell growth inhibitors and methods of using such inhibitors in treating certain diseases or disorders, e.g., tumor, cancer and bacterial infection, are also provided.

The invention disclosed in this application was funded in part by NIH grants RO1 GM52847 and RO1 GM46330. The United States government has certain rights in the invention disclosed and claimed in this application

### FIELD OF THE INVENTION

The present invention generally relates to cell growth modulators, methods of screening for such modulators and methods of using such modulators. In particular, the present invention provides a method of identifying a modulator of cell growth, which method comprises: a) assessing activity of a site-specific DNA recombinase or a type I DNA topoisomerase in the presence of a test substance; b) assessing activity of said site-specific DNA recombinase or said type I DNA topoisomerase in the absence of said test substance; and c) comparing said activities assessed in steps a) and b), whereby a difference in said activity assessed in step a) and said activity assessed in step b) indicates that said test substance modulates cell growth. Peptide cell growth inhibitors and methods of using such inhibitors in treating certain diseases or disorders, e.g., tumor, cancer and bacterial infection, are also provided.

### BACKGROUND OF THE INVENTION

The study of biochemical reactions is essential for our understanding of gene expression, DNA replication, DNA repair, cell division, and myriad other reactions that occur within living cells. Dissecting biochemical mechanisms relies on the ability to divide pathways into constituent steps, achieved by stabilizing transition-state intermediates or blocking specific steps in the pathway. However, studying intermediates and assessing the rate-limiting step has been very difficult in reactions which do not require external cofactors, and which are very efficient and highly reversible. One example of such reactions is site-specific recombination.

Site-specific recombination reactions are widespread in nature and are used to control gene expression, amplify episome copy number, create genetic diversity, and separate chromosomes at bacterial cell division (Landy, 1989; Nash, 1996). Phage λ integrase (Int), a member of a large family of tyrosine recombinases (Esposito & Scocca, 1997; Nunes-Duby et al., 1998), integrates the phage genome into the host genome to generate a lysogen or excises the prophage, allowing it to resume lytic growth. These integrative and excisive recombination reactions are unidirectional (the products differ from the substrates) and involve accessory factors encoded by the phage (e.g., Excisionase (Xis)) and by the host (e.g., the Integration Host Factor (IHF)).

The tyrosine recombinases mediate catalysis by attacking the phosphodiester backbone of one DNA strand from each partner substrate using a tyrosine residue, making a transient 3′ protein-DNA covalent bond (FIG. 1). Strand exchange between DNA partners follows, and a transesterification reaction mediated by the free 5′ OH group displaces the protein from the DNA to generate a Holliday junction (HJ). A second set of DNA cleavage, strand exchange, and ligation steps occurring at the bottom strands of each substrate DNA resolves the HJ into two recombinant products. The strand exchanges use homology as a way to test the suitability of DNA substrates: if the substrates are not identical in a 7 base pair region between the loci of strand cleavage and ligation, the reaction is quickly reversed to starting substrates (Burgin & Nash, 1995; Kitts & Nash, 1987; Nunes-Duby et al., 1995). This reversibility together with the fact that these reactions require no external high energy cofactors for binding or catalysis have made it difficult to identify the rate limiting step and to analyze reaction intermediates.

One approach to blocking Int-mediated recombination at intermediate stages has been to use modified DNA substrates of 3 basic types. First, heterologies between the two substrates block efficient strand exchange and slightly increase the amount of protein-DNA covalent intermediates by apparently inhibiting ligation (Kitts & Nash, 1987; Richet et al., 1988; Nash & Robertson, 1989; Burgin & Nash, 1995). Second, nicking the DNA phosphodiester backbone near the cleavage loci also blocks the ligation step due to diffusion of a 3-base oligomer whose base pairing is destabilized (Nunes-Duby et al., 1987; Pargellis et al., 1988). Third, phosphorothioate, phosphonate and phosphoramidate modifications (i.e., modifications of DNA backbone atoms) block the cleavage step (Kitts & Nash, 1987; Kitts & Nash, 1988; Burgin & Nash, 1995; S. Robinson, G. Cassell, A. Burgin & A. Segall, unpublished results) while phosphorothiolate modifications block the ligation step (Burgin and Nash, 1995) in certain DNA substrates. While these nucleotide modifications have given crucial insights into the mechanism and structure of both tyrosine recombinases and topoisomerases (Kitts and Nash, 1987, 1988; Richet et al., 1988; Nash and Robertson, 1989; Redinbo et al., 1998; Stewart et al., 1998), each of them has their limitations regarding the class of intermediates which accumulate. On one hand, heterologies do not lead to significant accumulation of intermediates, such as covalent protein-DNA intermediates or Holliday junctions, due to the reversibility of the reaction. On the other hand, DNA modifications can be easily introduced only into linear substrates, while integration requires a covalently-closed supercoiled molecule as one of the substrates.

A second approach to isolating intermediates has been to use Integrase mutants. The IntF mutant lacking the active site tyrosine (Y342F) does not cleave DNA (Pargellis et al., 1988), and has been used to address the issue of cis versus trans DNA cleavage (Han et al., 1993; Nunes-Duby et al., 1994). The drawback of the IntF mutant is that the absence of the tyrosine decreases the accumulation and/or stability of some intermediate complexes (Segall, 1998). The IntH mutant (IntE174K) accumulates intermediates at a higher level but the increase is quite modest (Kitts & Nash, 1987; 1988). Int mutants which have hypertopoisomerase activity have been isolated and are being studied (Han et al., 1994).

Biochemical reactions mediated by some recombinases and DNA topoisomerases are associated with certain diseases or disorders and the recombinases and DNA topoisomerases involved in such diseases or disorders have diagnostic and/or therapeutic values. For example, application of the Cre recombinase/loxP system enhances antitumor effects in cell type-specific gene therapy against carcinoembryonic antigen-producing cancer (Kijima et al., Cancer Res., 59(19):4906-11 (1999)). African-American race and antibodies to topoisomerase I are independent risk factors for scleroderma lung disease (Greidinger et al., Chest, 114(3):801-7 (1998)). Assays for anti-topoisomerase I antibodies and anticentromere antibodies complement the findings from nailfold capillaroscopy in providing useful prognostic information in Raynaud's disease (Weiner et al., Arthritis Rheum., 34(1):68-77 (1991)).

As pathogenic bacteria become resistant to the currently available antibiotics, new ones must be developed. Lack of new antibiotics will mean return to the health environment in the pre-antibiotic era. Expansion of the antibiotic repertory should include exploring new families of enzymes which can serve as targets against antibiotics. By the same token, the fight against cancer should include the expansion of the repertory of cancer therapeutics.

Accordingly, better understanding of certain enzymes such as site-specific recombinase and type I DNA topoisomerases will allow researchers to gain insight into the physiological or pathological mechanisms and identify new therapeutic targets for diseases or disorders associated with uncontrolled and/or undesired cell growth such as tumor, cancer and bacterial infection. New methods for studying the enzymes involved in these diseases and methods for screening for modulators of cell growth are needed. The present invention addresses these and other related needs in the art.

### SUMMARY OF THE INVENTION

In one aspect, the present invention encompasses a method of identifying a modulator of cell growth, which method comprises: a) assessing activity of a site-specific DNA recombinase or a type I DNA topoisomerase in the presence of a test substance; b) assessing activity of said site-specific DNA recombinase or said type I DNA topoisomerase in the absence of said test substance; and c) comparing said activities assessed in steps a) and b); whereby a difference in said activity assessed in step a) and said activity assessed in step b) indicates that said test substance modulates cell growth.

In another aspect, the present invention encompasses an isolated peptide for inhibiting a tyrosine recombinase, which isolated peptide has the following formulas:

1) a peptide having the following formula:

(Xaa1-Xaa2-Xaa3-Xaa4)n

wherein Xaa1 is an aromatic or a basic amino acid residue, Xaa2 is Ser, Cys, an aromatic or a basic amino acid residue, Xaa3 is any amino acid residue, Xaa4 is Ser, Cys, an aromatic or a basic amino acid residue, wherein each of Xaa1, Xaa2, Xaa3, or Xaa4 can be a D or L amino acid residue and wherein n is an integer ranging from 1 to 10;

2) a peptide having the following formula:

(Xaa1-Xaa2-Xaa3-Xaa4-Xaa5)n

wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, wherein each of Xaa1, Xaa2, Xaa3, Xaa4 or Xaa5 can be a D or L amino acid residue and wherein n is an integer ranging from 1 to 10; or

3) a peptide having the following formula:

(Xaa1-Xaa2-Xaa3-Xaa4-Xaa5-Xaa6)n

wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, Xaa6 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

In still another aspect, the present invention encompasses an isolated peptide for inhibiting a tyrosine recombinase or a type I DNA topoisomerase, which isolated peptide has the following formulas:

1) a peptide having the following formula:

(Xaa1-Xaa2-Xaa3-Xaa4)n

wherein each of Xaa1 and Xaa2 is an aromatic amino acid residue, Xaa3 is Ser, Cys or an aromatic amino acid residue, Xaa4 is an aromatic or a basic amino acid residue, wherein each of Xaa1, Xaa2, Xaa3, or Xaa4 can be a D or L amino acid residue and wherein n is an integer ranging from 1 to 10.

2) a peptide having the following formula:

(Xaa1-Xaa2-Xaa3-Xaa4-Xaa5)n

wherein Xaa1 is a basic amino acid residue, each of Xaa2 and Xaa3 is an aromatic amino acid residue, Xaa4 is Ser, Cys or an aromatic amino acid residue, Xaa5 is an aromatic or a basic amino acid residue, wherein each of Xaa1, Xaa2, Xaa3, Xaa4 or Xaa5 can be a D or L amino acid residue and wherein n is an integer ranging from 1 to 10; and

3) a peptide having the following formula:

(Xaa1-Xaa2-Xaa3-Xaa4-Xaa5-Xaa6)n

wherein Xaa1 is a basic amino acid residue, each of Xaa2 and Xaa3 is an aromatic amino acid residue, each of Xaa4 and Xaa6 is Ser, Cys or an aromatic amino acid residue, Xaa5 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

In yet another aspect, the present invention encompasses a method for inhibiting cell growth in a subject, which method comprises administering to a subject, to which such inhibition is desirable, an effective amount of an inhibitor of a site-specific DNA recombinase or a type I DNA topoisomerase, whereby cell growth is inhibited.

### BRIEF DESCRIPTION OF THE DRAWINGS

FIG. 1 depicts catalytic events mediated by Int in integrative and excisive recombination. The Int protein together with appropriate accessory factors juxtaposes the two recombination substrates in a synaptic complex. The active site tyrosine of each Int monomer attacks a specific phosphodiester linkage and forms a transient covalent 3′-phosphotyrosyl bond between the enzyme and the top strand of each DNA substrate. Ligation occurs when the free 5′OH from a partner substrate (or from the original substrate) acts as a nucleophile at this phosphotyrosyl linkage. Since the two DNA strands of each substrate are cleaved independently, a Holliday junction is generated during recombination. The Holliday junction is resolved by a repetition of the previous DNA cleavage, strand exchange and ligation steps on the bottom strand of each DNA substrate, resulting in two recombinant DNA molecules.

FIG. 2 illustrates a strategy for deconvolution of the SPCLs. In step 1, one position in the hexapeptide is fixed (denoted by 0) with one of the 20 amino acids and the remaining positions (denoted by X) are mixtures of 19 amino acids (all except cysteine). In this step, 20 mixtures/single position were generated. 120 mixtures total, representative of 2.47×10⁶ peptides/mixture, were tested. Most potent mixtures were ranked by dose response titrations at each fixed position. The best candidates from step 1 are chosen for inclusion in mixtures containing two defined positions. The same complexity of peptides can be found in libraries used in step 2 of the deconvolution process; these are known as dual-defined position libraries because each hexapeptide contains two fixed positions while the remaining 4 positions contain one of 19 amino acids. Because of this, a set of 400 dual-defined libraries in which positions 1 and 2 are fixed represent the same complexity as 40 single-defined libraries in which either position 1 or position 2 is fixed. However, individual dual-defined libraries are much less complex than each single-defined library. Thus, in step 2, 400 mixtures/pair of positions were generated. 1,200 mixtures total, representative of 132,000 peptides/mixture, were generated. 50-60 mixtures have been tested for desired phenotype(s). Most potent mixtures were ranked by dose response titrations. The best candidates from step 2 are chosen for inclusion in individual peptides. Finally, step 3 entails testing peptides of completely defined sequence. 7 or 12 individual peptides were synthesized and peptides were ranked by dose response titrations.

FIG. 3 depicts a representation of the structure of reaction intermediates and real gel figures are not shown here. The CPD (covalent protein-DNA) intermediates are sensitive to proteinase K (lane 2 versus lane 3 and lane 4 versus lane 5). The faster of the CPD complexes is formed with labeled substrate DNA (lane 4), while the slower CPDs is formed with recombinant products.

FIG. 4 depicts examples of the effect on recombination of single- and dual-defined peptide libraries. In the top (first position defined) and middle (second position defined) panels, recombination reactions were treated with a final concentration of 1 mg/ml total peptides. The amino acid in the fixed position is denoted along the X axis. Values of % recombination (top and middle panels) were normalized to the extent of recombination in untreated reactions. The mixtures representing the amino acids which were chosen for the 2nd step of deconvolution are marked (♦). These mixtures were chosen not only on the basis of the data shown here, but also on the results of dose response assays with the top 9-12 candidate mixtures at each position at 0.33 mg/ml and 0.11 mg/ml final concentration. In the bottom panel, the example given is of the library with the first two positions defined. Recombination reactions were treated with a final concentration of 11 μg/ml total peptides. Values of % inhibition were calculated based on the amount of recombination in untreated reactions (%recombination+peptide/% recombination−peptide×100%). The mixtures that were most potent at blocking recombination after dose response assays are marked (♦).

FIG. 5 depicts examples of the effect on accumulation of Holliday junctions of single- and dual-defined position peptide libraries. In the top (first position defined) and middle (second position defined) panels, recombination reactions were treated with a final concentration of 1 mg/ml total peptides. The amino acid in the fixed position is denoted along the X axis. Values of % Holliday junctions were calculated as the % of total counts in the reaction present as HJs on a gel such as the one shown in FIG. 3A. The mixtures representing the amino acids which were chosen for the 2nd step of deconvolution are marked (♦). These mixtures were chosen not only on the basis of the data shown here, but also on the results of dose response assays with the top 9-12 candidate mixtures at each position at 0.33 mg/ml and 0.11 mg/ml concentration. In the bottom panel, the example given is of the library with the first two positions defined. Recombination reactions were treated with a final concentration of 11 μg/ml total peptides. The mixtures which were most potent at accumulating HJs after dose response assays are marked (♦).

FIG. 6A depicts dose response titrations of peptides that inhibit recombination early in the pathway. Specific peptides were added to recombination reactions at the concentrations specified. Peptide 59 is the most potent, with an IC₅₀ of 0.02 μM, and exerts an almost complete block on recombination at 1 μM. Percent recombination was determined as the % of total counts in the reaction present as recombinant product bands on a gel like the one shown in FIG. 3A. 6B depicts dose response titrations of peptides which cause the accumulation of Holliday junctions. Specific peptides were added to recombination reactions at the concentrations specified. Peptide 52 is the most potent, with an IC₅₀ of 0.2 μM. The most HJs accumulate at about 2 μM. The amount of HJs that accumulates at higher peptide concentrations may stay the same or decrease because the peptides may have filled all available binding sites or may begin to block DNA cleavage at these concentrations. Percent HJs was determined as the % of total counts in the reaction present as HJs on a gel like the one shown in FIG. 3A.

FIG. 7 depicts determination of the importance of specific amino acid R groups at each position of the hexapeptides. 7A. Alanine scan of peptide 59 (each position of the peptide was individually substituted with alanine) and replacement of lysine with arginine at position 1. The effects of peptide 59 on % recombination are shown at two different peptide concentrations. 7B. Alanine scan of peptide 52 and the effect of replacing the carboxy-terminal amide with a carboxyl group. The effects of peptides with alanine or carboxyl substitutions were expressed as a percentage of the effect of peptide 52 on accumulation of HJs, which was defined as 100%.

FIG. 8 depicts effect of peptides on recombination (peptide 59—panel A; peptide 52—panel B) and on Holliday junction accumulation (peptide 52-panel B) as a function of time. Recombination reactions were untreated or treated with peptide 59 at 1 μM final concentration or with peptide 52 at 10 μM final concentration, and stopped with SDS-containing loading buffer after the specified length of time. The % recombination or % HJs were quantitated as described above.

FIG. 9 depicts DNA substrates and proteins necessary for bacteriophage λ integrative and excisive recombination.

FIG. 10 depicts effect of peptide inhibitors on bent-L recombination. A. Recombination reactions were assembled as specified in Materials and Methods, containing one double end-labeled substrate (Sub) and a longer unlabeled substrate in the presence of 100 ng salmon sperm DNA. Recombinant products are labeled Rec, covalent protein-DNA intermediates are labeled CPD, and Holliday junctions are labeled HJ. Peptide was added at the specified concentrations. Recombination extents were normalized to the amount of recombination in untreated reactions and expressed as relative % recombination. B. Comparison of the dose response titrations of peptide KWWCRW (closed circles) and peptide KWWWRW (closed squares) in bent-L recombination. The IC₅₀ value for peptide KWWCRW is 0.02 μM, while for peptide KWWWRW it is roughly 0.04 μM. C. The effect of peptide KWWCRW (closed circles) and peptide KWWWRW (closed squares) on accumulation of Holliday junctions during bent-L recombination. The % HJ were calculated as the fraction of total counts in each reaction×100%.

FIG. 11 depicts effect of peptide KWWCRW (diamonds) and peptide KWWWRW (squares) on the remaining 3 pathways of Int-mediated recombination. Recombination extents were normalized to the amount of recombination in untreated reactions and expressed as relative % recombination. A. Effect of peptides on integrative recombination (IC₅₀ values for both peptides are roughly 0.2 μM). Reactions were assembled as for bent-L recombination except that they were incubated at room temperature, the substrates were a supercoiled plasmid (4.8 kb) carrying attP and ³²P labeled 91 bp PCR fragment encoding attB. B. Effect of peptides on excisive recombination (IC₅₀ values for both peptides of about 1.1 μM). The recombination substrates were PCR fragments encoding the attL (³²P-labeled) and attR sites. Reactions contained 50 NM Xis in addition to Int and IHF (37 nM), as well as 100 ng salmon sperm DNA, and were incubated at room temperature. C. Effect of peptides on straight-L recombination (IC₅₀ values for peptide KWWCRW is 0.06 μM, while for peptide KWWWRW is slightly over 0.1 μM). Reactions were assembled as for bent-L recombination except that they were incubated at room temperature; the substrates were two PCR fragments, one of which was ³²P-labeled and 187 bp, the other of which was unlabeled and 496 bp.

FIG. 12 depicts effect of salmon sperm DNA concentration on the effects of peptide inhibitors on bent-L recombination. The absolute % recombination in these reactions was about the same with 100 ng salmon sperm DNA (9.3-11.7%) as with 300 ng salmon sperm DNA (10.25-11.6%), and somewhat higher with 1 μg salmon sperm DNA (12.2-14.3%).

FIG. 13 depicts peptide inhibition of single-turnover DNA cleavage catalyzed by vaccinia topoisomerase. The structure of the CCCTF-containing suicide substrate is shown, with the cleavage site is indicated by the arrow. The DNA was 5′ ³²P-labeled on the scissile strand. Cleavage reaction mixtures (20 pl) contained 50 mM Tris-HCl (pH 7.5), 0.1 pmol of 18-mer/30-mer DNA substrate, 0.5 pmol of vaccinia topoisomerase, and peptides as specified. Mixtures containing buffer and DNA were preincubated with the peptides for 10 min at 37° C. in the absence of topoisomerase. The cleavage reactions were initiated by adding topoisomerase and quenched after 10 s at 37° C. by adding SDS to 1% final concentration. The denatured samples were electrophoresed through a 10% polyacrylamide gel containing 0.1% SDS. The extent of covalent adduct formation (expressed as the % of input labeled DNA transferred to the topoisomerase polypeptide) was quantitated by scanning the gel with a Phosphorimager and is plotted as a function of the concentration of peptide in the reaction mixtures. 7A. Titration of KWWWRW and WKHYNY 7B. Titration of KWWCRW and WCHYNY.

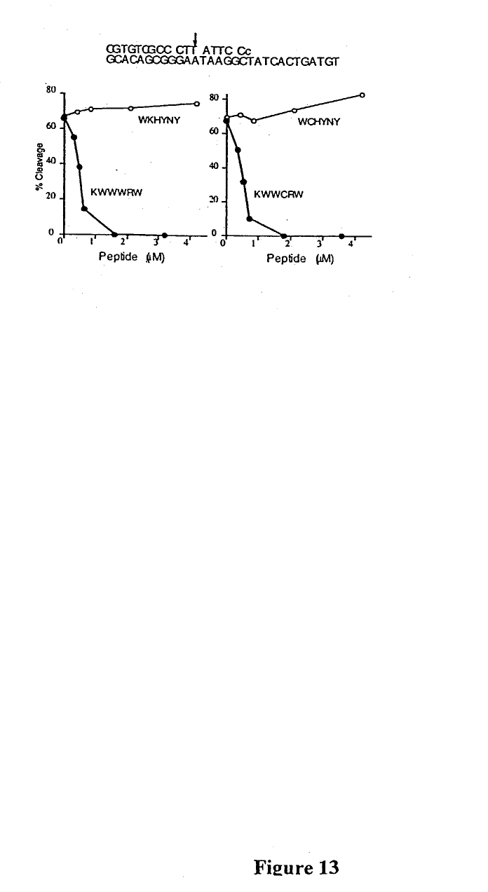

FIG. 14 depicts peptide effects on the kinetics of DNA cleavage by vaccinia topoisomerase. Reaction mixtures containing (per 20 pl)50 mM Tris HCl (pH 7.5), 0.1 pmol of 18-mer/30-mer DNA substrate, 0.5 pmol of vaccinia topoisomerase, and peptides as specified were incubated at 37° C. The reactions were initiated by the addition of enzyme to DNA (control) or to the preincubated DNA/peptide mixture. Aliquots (20 μl) were withdrawn at the times indicated and quenched immediately with SDS. Covalent adduct formation is plotted as a function of time.

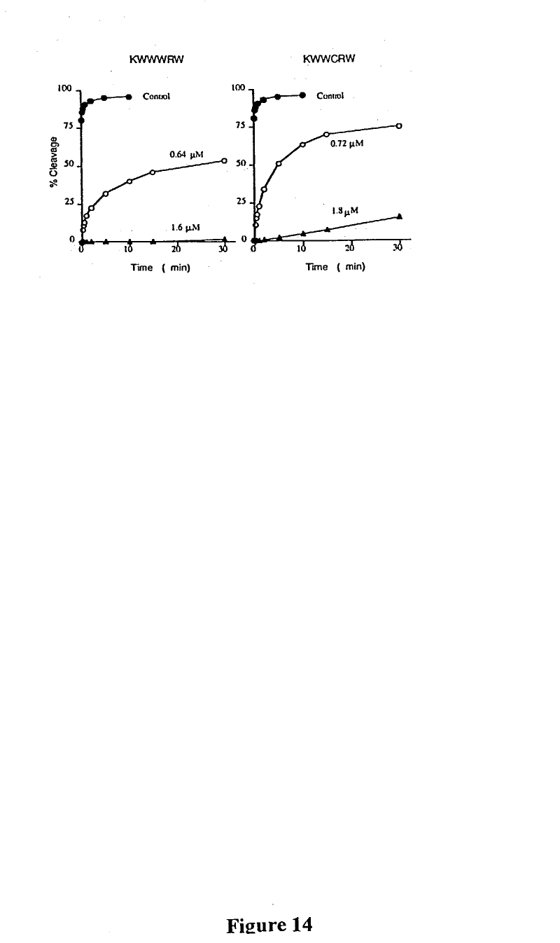

FIG. 15 depicts salt diminishes peptide potency in inhibiting vaccinia topoisomerase. Reaction mixtures (20 p.l) containing 50 mM Tris-HCl (pH 7.5), 0.1 pmol of 18-mer/30-mer substrate, 0.5 pmol of vaccinia topoisomerase, KWWCRW peptide as specified, and either 100 mM NaCl or r˜ added NaCl were incubated for 10 s at 37° C. The extent of covalent adduct formation is plotted as a function of peptide concentration.

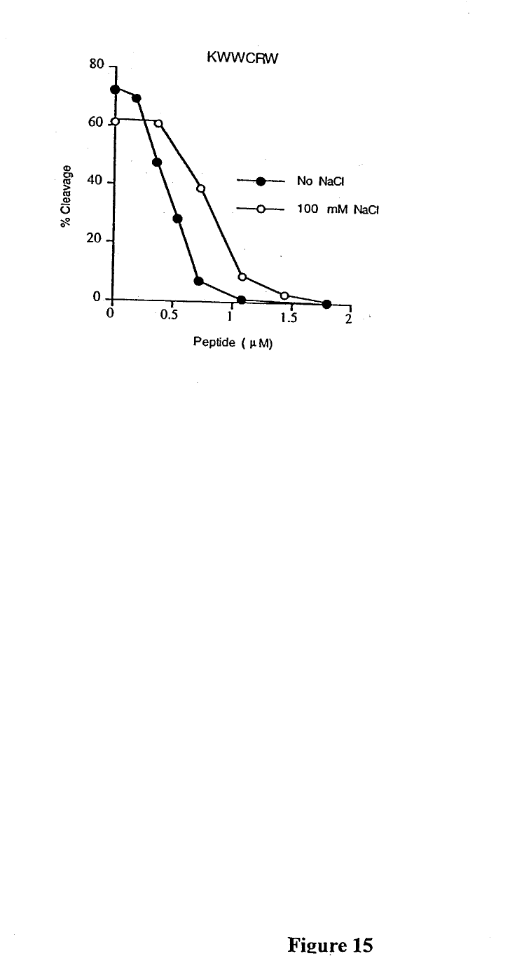

FIG. 16. A. Schem of excisive and bent-L recombination.attL and attR, which flank the integrated lambda prophage, site-specifically recombine to generate attP and attB in the presence of Int, IHF, and Xis. Two attL sites can recombine with each other in the bent-L pathway in the presence of Int and IHF; this recombination event is bidirectional. In vitro, the attL sites carry the tenP′1 mutation (see text). B The 7 bp overlap region is indicated, with −2 being the point of top strand cleavage and +4 the point of bottom strand cleavage (indicated by arrows). The key identifies Int and accessory protein binding sites. Schematic illustration of catalytic events of Int-mediated site-specific recombination is shown in FIG. 1.

FIG. 17. A. The hexameric peptide WKHYNY leads to accumulation of Holliday junctions in all four λsite-specific recombination pathways, with a wide range of effective concentration specific for each reaction. Bent-L recombination, solid squares. Integration, solid circles. Excision, open squares. Straight-L recombination, triangles. B Timecourses of excision reactions in the presence and absence of WKHYNY. C Timecourses of in vitro reactions in the presence and absence of WKHYNY in bent-L recombination. For both panesl B and C, recombinant products, circles; HJs, triangles; absence of peptide, solid symbols; presence of peptide, open symbols.

FIG. 18 depicts effect of peptide WKHYNY on DNA cleavage. A. Time course of resolution of excision HJs. HJs were isolated, and Int, IHF, and Xis were added in the presence or absence of peptide 52 (100 μM) and stopped at various timepoints (1, 5, 15, 30, 60, and 90 min). Absence of peptide, closed circles; presence of peptide, open squares. B. The extent of strand cleavage of attL. An attL site containing a phosphorothiolate modification at the point of top strand cleavage (attLS) was incubated with Int, IHF, and Xis, in the presence of attR and in the presence or absence of peptide WKHYNY. Absence of peptide, open squares; presence of peptides, closed diamonds.

FIG. 19 depicts representation of bimolecular complexes accumulate in excision in the presence of peptide WKHYNY and real gel figures are not shown here. Excision reactions were assembled and separated on a native gel. Without the peptide, the main complex seen is the attP recombinant product (the attB product is off this gel). With peptide, a new complex EX-HJC is seen.

FIG. 20 depicts comparison of the gel-based and microtiter-based screening assays for test substances that accumulate Holliday junction intermediates.

### DETAILED DESCRIPTION OF THE PREFERRED EMBODIMENTS

A. Definitions

Unless defined otherwise, all technical and scientific terms used herein have the same meaning as is commonly understood by one of ordinary skill in the art to which this invention belongs. All patents, applications, published applications and other publications and sequences from GenBank and other data bases referred to herein are incorporated by reference in their entirety.

As used herein, “assessing” refers to quantitative and/or qualitative determination of the activity of a site-specific DNA recombinase or a type I DNA topoisomerase, e.g., obtaining an absolute value for the amount or concentration of the substrate, intermediate and/or product of the reaction mediated by the site-specific DNA recombinase or a type I DNA topoisomerase, and also of obtaining an index, ratio, percentage, visual or other value indicative of the level of the substrate, intermediate and/or product. Assessment may be direct or indirect and the chemical species actually detected need not of course be the substrate, intermediate and/or product itself but may, for example, be a derivative thereof or some further substance.

As used herein, “DNA recombination” refers to cross-over reaction between DNA sequences.

As used herein, “generalized DNA recombination” refers to cross-over reaction between homologous DNA sequences. Its critical feature is that the enzymes responsible of the recombination can use any pair of homologous sequences as substrates, although some types of sequences may be favored over others.

As used herein, “site-specific DNA recombination” refers to cross-over reaction between specific pairs of DNA sequences. The enzyme involved in this event cannot recombine other pairs of, whether homologous or nonhomologous, sequences, but act only on the particular pair of DNA sequences.

As used herein, “site-specific DNA recombinase” refers to an enzyme that catalyzes the site-specific DNA recombination. The term “site-specific DNA recombinase” also encompasses any functional fragment, analog, homolog, derivative or mutant that still substantially retain its catalytic activity.

As used herein, “tyrosine recombinase” refers to a site-specific DNA recombinase that mediates catalysis by attacking the phosphodiester backbone of one DNA strand from each partner substrate using a tyrosine residue, making a transient 3′ protein-DNA covalent bond. The term “tyrosine recombinase” also encompasses any functional fragment, analog, homolog, derivative or mutant that still substantially retain its catalytic activity.

As used herein, “DNA topoisomerase” refers to an enzyme that can change the linking number of DNA. The term “DNA topoisomerase” also encompasses any functional fragment, analog, homolog, derivative or mutant that still substantially retain its catalytic activity.

As used herein, “type I DNA topoisomerase” refers to an enzyme that cuts DNA one strand at a time. The term “type I DNA topoisomerase” also encompasses any functional fragment, analog, homolog, derivative or mutant that still substantially retain its catalytic activity.

As used herein, “substantially retain its activity” means that an enzyme analog, homolog, derivative or mutant retains at least 50% of its catalytic activity comparing to its wild-type counterpart. Preferably, the enzyme analog, homolog, derivative or mutant retains at least 60%, 70%, 80%, 90%, 95%, 99% or 100% of its catalytic activity comparing to its wild-type counterpart.

As used herein, “test substance” refers to a chemically defined compound (e.g., organic molecules, inorganic molecules, organic/inorganic molecules, proteins, peptides, nucleic acids, oligonucleotides, lipids, polysaccharides, saccharides, or hybrids among these molecules such as glycoproteins, etc.) or mixtures of compounds (e.g., a library of test compounds, natural extracts or culture supernatants, etc.) whose effect on a site-specific DNA recombinase or a type I DNA topoisomerase is determined by the disclosed and/or claimed methods herein.

As used herein, “bioactive substance” refers to any substance that has been proven or suggested to have the ability of affecting a biological process or system. For example, any substance that are know to have prophylactic, therapeutic, prognostic or diagnostic value is considered a bioactive substance.

As used herein, “an effective amount of a compound for treating a particular disease” refers to an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein, “plant” refers to any of various photosynthetic, eucaryotic multicellular organisms of the kingdom Plantae, characteristically producing embryos, containing chloroplasts, having cellulose cell walls and lacking locomotion.

As used herein, “animal” refers to a multi-cellular organism of the kingdom of Animalia, characterized by a capacity for locomotion, nonphotosynthetic metabolism, pronounced response to stimuli, restricted growth and fixed bodily structure. Non-limiting examples of animals include birds such as chickens, vertebrates such as fish and mammals such as mice, rats, rabbits, cats, dogs, pigs, cows, ox, sheep, goats, horses, monkeys and other non-human primates.

As used herein, “infection” refers to invasion of the body of a multi-cellular organism with organisms that have the potential to cause disease.

As used herein, “infectious organism” refers to an organism that is capable to cause infection of a multi-cellular organism. Most infectious organisms are microorganisms such as viruses, bacteria and fungi.

As used herein, “bacteria” refers to small prokaryotic organisms (linear dimensions of around 1 μm) with non-compartmentalized circular DNA and ribosomes of about 70S. Bacteria protein synthesis differs from that of eukaryotes. Many antibacterial antibiotics interfere with bacteria proteins synthesis but do not affect the infected host.

As used herein, “eubacteria” refers to a major subdivision of the bacteria except the archaebacteria. Most Gram-positive bacteria, cyanobacteria, mycoplasmas, enterobacteria, pseudomonas and chloroplasts are eubacteria. The cytoplasmic membrane of eubacteria contains ester-linked lipids; there is peptidoglycan in the cell wall (if present); and no introns have been discovered in eubacteria.

As used herein, “archaebacteria” refers to a major subdivision of the bacteria except the eubacteria. There are three main orders of archaebacteria: extreme halophiles, methanogens and sulphur-dependent extreme thermophiles. Archaebacteria differs from eubacteria in ribosomal structure, the possession (in some case) of introns, and other features including membrane composition.

As used herein, “fungus” refers to a division of eucaryotic organisms that grow in irregular masses, without roots, stems, or leaves, and are devoid of chlorophyll or other pigments capable of photosynthesis. Each organism (thallus) is unicellular to filamentous, and possesses branched somatic structures (hyphae) surrounded by cell walls containing glucan or chitin or both, and containing true nuclei.

As used herein, “disease or disorder” refers to a pathological condition in an organism resulting from, e.g., infection or genetic defect, and characterized by identifiable symptoms.

As used herein, “neoplasm” (neoplasia) refers to abnormal new growth, and thus means the same as tumor, which may be benign or malignant. Unlike hyperplasia, neoplastic proliferation persists even in the absence of the original stimulus.

As used herein, “cancer” refers to a general term for diseases caused by any type of malignant tumor.

As used herein, “antibiotic” refers to a substance either derived from a mold or bacterium or organically synthesized, that inhibits the growth of certain microorganisms without substantially harming the host of the microorganisms to be killed or inhibited.

For clarity of disclosure, and not by way of limitation, the detailed description of the invention is divided into the subsections that follow.

B. Methods of Identifying Peptide Modulators

In one aspect, the present invention encompasses a method of identifying a modulator of cell growth, which method comprises: a) assessing activity of a site-specific DNA recombinase or a type I DNA topoisomerase in the presence of a test substance; b) assessing activity of said site-specific DNA recombinase or said type I DNA topoisomerase in the absence of said test substance; and c) comparing said activities assessed in steps a) and b), whereby a difference in said activity assessed in step a) and said activity assessed in step b) indicates that said test substance modulates cell growth.

The present method can be used to screen for cell growth enhancers and/or inhibitors. In one specific embodiment, the activity assessed in step a) is more than the activity assessed in step b), which indicates that said test substance enhances cell growth. In another specific embodiment, the activity assessed in step a) is less than the activity assessed in step b), which indicates that said test substance inhibits cell growth.

The cell growth modulator can be identified by its ability to affect overall efficiency or equilibrium of an intermediate of the DNA recombination mediated by the site-specific DNA recombinase or the type I DNA topoisomerase.

Any site-specific DNA recombinase or type I DNA topoisomerase can be used in the present screening method. For example, a tyrosine recombinase or other types of site-specific DNA recombinases such as Cre (Drago et al., J. Neurosci., (1998) 18(23):9845-57), Bacillus subtilis sporulation gene spoIVC (Sato et al., J. Bacteriol., (1990) 172(2):1092-8) and rci (Kubo et al., Mol. Gen. Genet., (1988) 213(1):30-5) can be used. Preferably, the site-specific DNA recombinase used is a tyrosine recombinase. Eukaryotic or prokaryotic tyrosine recombinase can be used. For example, a tyrosine recombinase derived from a human, an animal, e.g., a mammal or an insect, a plant and a fungus, e.g., yeast, species can be used. Preferably, the prokaryotic tyrosine recombinase used is a bacterial tyrosine recombinase. The bacterial tyrosine recombinase can be an eubacterial or archaebacterial tyrosine recombinase, a gram positive or gram negative bacterial tyrosine recombinase. More preferably, the bacterial tyrosine recombinase is derived from an enteric pathogenic bacterium, or is derived from a SALMONELLA, a SHIGELLA, a STAPHYLOCOCCUS, a STREPTOCOCCUS and a BACILLUS species or is an E. coli. tyrosine recombinase. Also more preferably, the bacterial tyrosine recombinase is a XerC, a XerD (Spiers and Sherratt, Mol. Microbiol., (1999) 32(5): 1031-42), a Flp site-specific recombinase (Lee et a., J. Mol. Biol., (2000) 296(2):403-19), or a homolog thereof. In a specific embodiment, XerC with the following GenBank accession numbers can be used: AF033498 (Proteus mirabilis), AF028736 (Serratia marcescens), U92525 (Salmonella typhimurium), X84261 (L. leichmannii) and M38257 (Escherichia coli). In another specific embodiment, XerD with the following GenBank accession numbers can be used: AF 18839 (Staphylococcus aureus), AF033497 (Proteus mirabilis), AF146614 (Erwinia carotovora), AF093548 (Staphylococcus aureus) and U92524 (Salmonella typhimurium). Phage integrase, e.g., λ, phi, 80, P22, P2, 186, P4 and P1 phage integrase can be used. Preferably, phage λ integrase (Int) or a homolog thereof, is used.

Any type I DNA topoisomerase, including a type IA or type IB DNA topoisomerase, can be used in the present method. Preferably, the type IA DNA topoisomerase is E. coli topoisomerase I (TopA) or a homolog thereof. For example, TopA with the following GenBank accession numbers can be used: L35043 (Mycoplasma gallisepticum), U11862 (Human), U20964 (Haemophilus influenzae), U97022 (Fervidobacterium islandicum) and U11863 (Human). Also preferably, the type IB DNA topoisomerase is vaccinia virus topoisomerase or a homolog thereof. For example, vaccinia virus topoisomerase with the following GenBank accession number can be used: L13447 (Vaccinia virus).

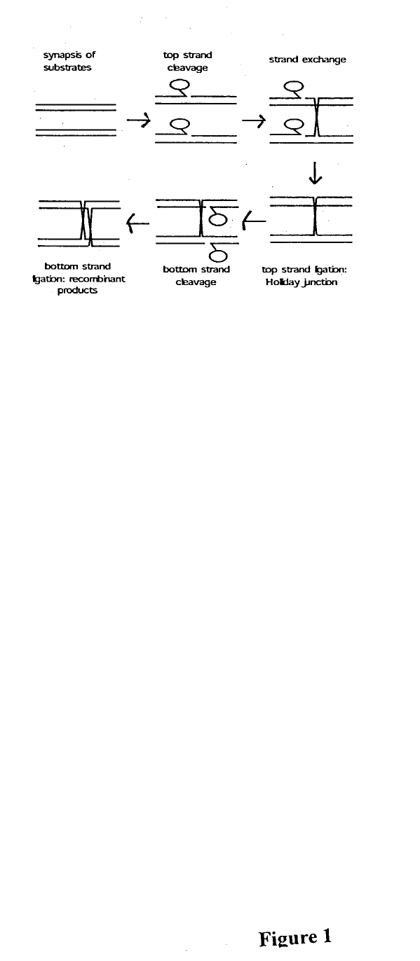

Tyrosine recombinases are a large class of enzymes with many biological functions. Once set of these enzymes, the integrases, are used by bacterial viruses (phages) to integrate their genomes into the chromosomes of their bacterial hosts. A related set of enzymes, exemplified by the XerC and XerD enzymes of E. coli, are necessary for the appropriate segregation of bacterial chromosomes to daughter cells. These enzymes are present in all bacterial cells examined, including gram+ and gram− cells (Sirois and Szatmari, 1995; Sciochetti et al., 1.999). Like other tyrosine recombinases, the Xer proteins carry out recombination using a type I topoisomerase mechanism by two successive rounds of strand nicking, exchange and strand sealing reactions. (FIG. 1) The active site residue is also a tyrosine which makes a covalent bond to DNA to leave a free 3″ hydroxyl group, like the eukaryotic type IB topoisomerases to which they are structurally related but not related by amino acid sequence (Cheng et al., 1998; Redinbo et al., 1999). Obligate intermediates of these reactions are covalent enzyme-DNA complexes and an unique structure called the Holliday junction; whereas type I topoisomerases also generate enzyme-DNA covalent complexes, they do not generate Holliday junctions as part of their mechanistic cycle. When either one of the Xer proteins or their target site in the bacterial chromosome are mutated, E. coli cells are unable to efficiently segregate sister chromosomes to daughter cells; instead, dimeric chromosomes remain stuck at the division point and prevent the septum from being completed. A large proportion of cells with Xer defects are anucleate and the viability of the culture is reduced drastically. Xer defects can be corrected by mutations in the RecA protein, the central protein in homologous recombination. However, the RecA protein is essential for the survival of pathogens in their hosts (Buchmeier et al., 1993), since homologous recombination is essential for the repair of DNA breaks induced by oxidative damage.

The Xer enzymes are good and untapped targets for screening for broad spectrum antibiotic compounds. Three types of inhibitors might be envisioned, based on the mechanism of these enzymes; 1) inhibitors of DNA cleavage; 2) inhibitors of religation; and 3) inhibitors of resolution of the Holliday junction intermediates. Because of the structural and mechanistic similarity between eukaryotic topoisomerases and tyrosine recombinases, the fist two types of inhibitors might cross-react with the mammalian topoisomerases and thus demonstrate unacceptable toxic side effects. A class of cancer therapeutics based on the natural product camptothecin are inhibitors of DNA religation, and in fact are cytotoxic (but acceptable risk for cancer patients). In contrast, inhibitors of the third type should (and do not; see below) inhibit topoisomerases, since these enzymes do not generate Holliday junction intermediates.

Accordingly, when a tyrosine recombinase is screened against in order to identify a cell growth inhibitor, any of its activity, including DNA strand cleavage activity, DNA strand religation activity and Holliday junction intermediate resolution activity, can be screened against. Preferably, especially when screening for an antibiotic, the tyrosine recombinase activity to be screened against is the Holliday junction intermediate resolution activity.

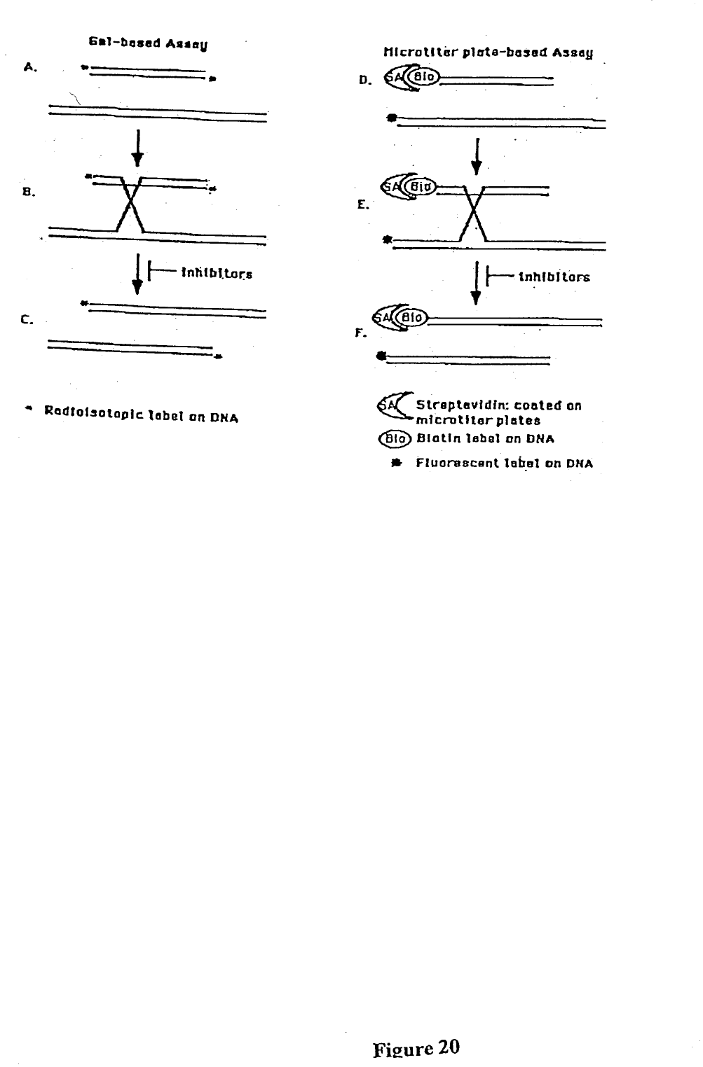

The Holliday junction intermediate resolution activity of a tyrosine recombinase can be screened against with suitable methods. In one specific embodiment, the Holliday junction intermediate resolution activity is assayed by conducting a tyrosine recombinase mediated recombination between two different-sized DNA duplexes, only one of said DNA duplexes is detectably labeled and successful recombination results in a detectably labeled DNA duplex with a size that is distinct from each of the original DNA duplexes, and assessing presence or amount of the Holliday junction intermediate which is resistant to protease digestion and migrates electrophoretically slower than said original DNA duplexes, said resulting recombinant DNA duplex and any covalent protein-DNA complex, whereby a test substance that increases the presence or amount of said Holliday junction intermediate indicates that said test substance inhibits the Holliday junction intermediate resolution activity of the tyrosine recombinase. For example, bacteriophage lambda Int-mediated recombination can use recombination between two DNA molecules, one radioisotopically labeled at both ends of the DNA, the other entirely unlabeled but of different size than the labeled molecule (FIG. 20A). Recombination between the two DNA molecules will result in 2 products, each radiolabeled at one end of the DNA and of unique size distinct from the labeled substrate DNA (FIG. 20C). Intermediates of the reaction can be followed by their unique properties. Covalent protein-DNA complexes (CPD) migrate more slowly than free DNA during electrophoresis due to the added mass of the protein and are resistant to protein denaturation by SDS or other protein detergents or denaturants, or any agents that do not reverse the covalent bond between the DNA and the protein. These complexes, however, are sensitive to and destroyed by general protease enzymes such as protease K. The Holliday junction also migrates more slowly than free substrate DNA and more slowly than the CPDs, because the fact that it contains four strands of DNA (from the two DNA substrates) rather than two (FIG. 20B). Because it contains no protein component, it is resistant to protease K. Thus peptides that stabilize the Holliday junction and prevent DNA cleavage lead to accumulation of this specific complex.

In another specific embodiment, the Holliday junction intermediate resolution activity is assayed by conducting a tyrosine recombinase mediated recombination between a DNA duplex that is capable of attaching to a solid surface and a DNA duplex that is detectably labeled, and assessing presence or amount of the Holliday junction intermediate which is both attached to said solid surface and is detectably labeled, whereby a test substance that increases the presence or amount of said Holliday junction intermediate indicates that said test substance inhibits the Holliday junction intermediate resolution activity of the tyrosine recombinase. For example, such assay can be conducted in a microtiter plate-based, high throughput assay format: by taking advantage of: 1) extremely high-affinity, extremely stable biotin-streptavidin interactions; 2) the ability to specifically introduce biotin into DNA and to coat microtiter plates with streptavidin; and 3) the ability to fluorescently label DNA. Each DNA substrate molecule is labeled at one end, e.g., during its synthesis by PCR, with either a biotin or a fluorescent group. Recombination reactions can be performed in 96- or 384-well microtiter plates which are coated with streptavidin (or avidin). All other necessary reagents for recombination are added as well as compounds to be tested for accumulation of Holliday junctions. At the beginning of the reaction, the biotin-labeled molecule will react with the streptavidin coating the plate. As recombination proceeds, the fluorescently labeled molecule will be joined to the same DNA as the biotin label as the Holliday junction forms (FIG. 20E), then will be separated from the biotin-labeled DNA as the Holliday junction is resolved into products (FIG. 20F). If the microtiter plate is washed with buffer containing a small amount of detergent, e.g., 0.1% SDS, or other protein denaturant, the only fluorescently-labeled DNA remaining in the microtiter plate will be the small amount of Holliday junctions that accumulate during normal reactions, fewer than 2% of input substrates. This small amount of fluorescently-labeled DNA remaining in the plate will be increased by compounds that stabilize the Holliday junction. One possible drawback of this experimental set-up is that one might lose unstably-bound peptides. This problem can be fixed by incubating reactants together, e.g., for 30 minutes, with the test compounds, then adding another peptide that blocks DNA cleavage by tyrosine recombinase enzymes, e.g., KWWCRW (see following Sections E and G). It has been found that once the Holliday junction-accumulating peptides stabilize Holliday junctions, Int can be prevented from processing them by the cleavage-inhibiting peptide even in the absence of the original peptide if “washed away”.

In still another specific embodiment, the Holliday junction intermediate resolution activity is assayed by conducting a tyrosine recombinase mediated recombination between a DNA duplex with a first label and a DNA duplex with a second label, and assessing presence or amount of the Holliday junction intermediate which gives a detectable signal resulted from proximity of said first and second label in the Holliday junction and said detectable signal is detectably distinct from the signal of said first and second label, whereby a test substance that increases the presence or amount of said Holliday junction intermediate indicates that said test substance inhibits the Holliday junction intermediate resolution activity of the tyrosine recombinase. Preferably, the first label and the second label are components of a fluorescence resonance energy transfer (FRET) detection system. Any FRET detection system known in the art can be used in the present method. For example, the AlphaScreen™ system can be used. AlphaScreen technology is an “Amplified Luminescent Proximity Homogeneous Assay” method. Upon illumination with laser light at 680 nm, a photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity of the donor bead, by virtue of a biological interaction, the singlet-state oxygen molecules reacts with chemiluminescent groups in the acceptor beads, which immediately transfer energy to fluorescent acceptors in the same bead. These fluorescent acceptors shift the emission wavelength to 520-620 nm. The whole reaction has a 0.3 second half-life of decay, so measurement can take place in time-resolved mode. Other exemplary FRET donor/acceptor pairs include Fluorescein (donor) and tetramethylrhodamine (acceptor) with an effective distance of 55 Å; IAEDANS (donor) and Fluorescein (acceptor) with an effective distance of 46 Å; and Fluorescein (donor) and QSY-7 dye (acceptor) with an effective distance of 61 Å (Molecular Probes).

When an Int is screened against, an Int inhibitor, and hence the cell growth inhibitor, can be identified by its ability of decreasing overall efficiency of the Int-mediated recombination or its ability of accumulating or stabilizing a Holliday junction or synaptic intermediate.

Any substance can be used as the test substance in the present method. The test substance can be inorganic molecules such as ions, organic molecules or a complex thereof. Non-limiting examples of organic molecules include amino acids, peptides, proteins, nucleosides, nucleotides, oligonucleotides, nucleic acids, vitamins, monosaccharides, oligosaccharides, carbohydrates, lipids or other bioactive substance, or a complex thereof. Preferably, the test substance is a peptide or a mixture thereof. The peptides to be screened can be of any suitable length. The peptide length should be decided in view of the site-specific recombination reaction to be screened against and target proteins or enzymes involved in the recombination reaction. If necessary, the peptide length can be determined empirically. Normally, the length of the peptides can be from about 4 amino acid residues to about 60 amino acid residues. Preferably, the length of the peptides can be from about 4 amino acid residues to about 10 amino acid residues. More preferably, the length of the peptides can be from about 4 amino acid residues to about 6 amino acid residues.

The peptide, or mixtures thereof, used in the screening can be made by any methods known in the art. The peptides can be produced by chemical synthesis, recombinant production, or a combination thereof. Preferably, the peptides are produced by chemical synthesis (see e.g., Combinational Peptide Library Protocols, Vol. 87, Cabilly (Ed.), Humana Press, 1998). Also preferably, mixture-based synthetic combinatorial libraries are used in the screening and such libraries can be made by methods known in the art including the methods disclosed in Houghten et al., J. Med. Chem., 42(19):3743-78 (1999). If the mixture-based synthetic combinatorial libraries are used in the screening, the following method can be used, which method comprises: (a) screening a first mixture of peptides capable of causing a desired change in a biochemical reaction mediated by a site-specific DNA recombinase or type I DNA topoisomerase, wherein at least one defined amino acid residue is fixed at a known position on each of the peptides of the first mixture, and identifying at least one particular amino acid residue at the fixed known position in the first mixture of peptides that is capable of causing the desired change; (b) screening a second mixture of peptides capable of causing the desired change in the biochemical reaction mediated by a site-specific DNA recombinase or type I DNA topoisomerase, wherein at least two defined amino acids are fixed at known positions on each peptide from the second mixture, and wherein at least one amino acid and its sequence position corresponds to the amino acid and the sequence position of a peptide from the first mixture as identified in step (a); and (c) selecting at least one peptide from the second mixture that is capable of causing the desired change in the biochemical reaction mediated by a site-specific DNA recombinase or type I DNA topoisomerase. The screening method can further comprise a step of generating at least one new peptide selected in step (c), wherein the new peptide comprises the two defined amino acids of the selected peptide from the second mixture, said two defined amino acids having sequence positions corresponding to the sequence positions of the selected peptide from the second mixture.

The screening can be conducted in vivo or in vitro. Preferably, the initial screening is conducted by in vitro tests. Although the method can be used in screening a single peptide mixture at a time, the method is preferably used in a high-throughput format, i.e., a plurality of peptide mixtures are tested simultaneously. In addition, a combinatorial library can be used in the screening assays. Methods for synthesizing combinatorial libraries and characteristics of such combinatorial libraries are known in the art (See generally, Combinatorial Libraries: Synthesis, Screening and Application Potential (Cortese Ed.) Walter de Gruyter, Inc., 1995; Tietze and Lieb, Curr. Opin. Chem. Biol., 2(3):363-71 (1998); Lam, Anticancer Drug Des., 12(3):145-67 (1997); Blaney and Martin, Curr. Opin. Chem. Biol., 1(1):54-9 (1997); and Schultz and Schultz, Biotechnol. Prog., 12(6):729-43 (1996)).

Cell growth modulators identified according to the above-described screening methods are also encompassed in the present invention.

C. Cell Growth Inhibiting Peptides

In another aspect, the present invention encompasses cell growth inhibiting peptides. In a specific embodiment, the present invention encompasses an isolated peptide for inhibiting a tyrosine recombinase, which peptide has the following formula:

(Xaa1-Xaa2-Xaa3-Xaa4)n

wherein Xaa1 is an aromatic or a basic amino acid residue, Xaa2 is Ser, Cys, an aromatic or a basic amino acid residue, Xaa3 is any amino acid residue, Xaa4 is Ser, Cys, an aromatic or a basic amino acid residue, wherein each of Xaa1, Xaa2, Xaa3, or Xaa4 can be a D or L amino acid residue and wherein n is an integer ranging from 1 to 10. Preferably, Xaa1 is Trp, Arg or Tyr; Xaa2 is Trp, Lys, Arg or Cys; Xaa3 is Ala, His, Val, Arg, Trp, Tyr or Cys; and Xaa4 is Trp, Cys, Tyr, Arg or Phe. Exemplary peptides of this group include: 1) Trp-Lys-Ala-Tyr; 2) Trp-Lys-His-Tyr; 3) Trp-Lys-Val-Tyr, 4) Trp-Arg-Arg-Trp, 5) Trp-Arg-Trp-Tyr; 6) Trp-Arg-Arg-Cys; 7) Trp-Arg-Tyr-Arg, 8) Arg-Cys-Trp-Trp; 9) Arg-Cys-Cys-Tyr; and 10) Tyr-Trp-Cys-Tyr. The isolated peptide can further comprise a Met as the first N-terminal amino acid residue to facilitate recombinant production.

In another specific embodiment, the present invention encompasses an isolated peptide for inhibiting a tyrosine recombinase, which peptide has the following formula:

(Xaa1-Xaa2-Xaa3-Xaa4-Xaa5)n

wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, wherein each of Xaa1, Xaa2, Xaa3, Xaa4 or Xaa5 can be a D or L amino acid residue and wherein n is an integer ranging from 1 to 10. Preferably, Xaa1 is Trp, Arg or Tyr; Xaa2 is Trp, Lys, Arg or Cys; Xaa3 is Ala, His, Val, Trp, Arg, Cys or Tyr; Xaa4 is Trp, Cys, Tyr Phe or Arg; and Xaa5 is Gln, Pro, Cys, Arg or Trp. Exemplary peptides of this group include: 1) Trp-Lys-Ala-Tyr-Gln; 2) Trp-Lys-His-Tyr-Pro; 3) Trp-Lys-His-Tyr-Gln; 4) Trp-Lys-Val-Tyr-Pro; 5) Trp-Lys-Val-Tyr-Gln; 6) Trp-Lys-Ala-Tyr-Pro; 7) Trp-Arg-Arg-Trp-Cys; 8) Trp-Arg-Trp-Tyr-Cys; 9) Trp-Arg-Arg-Cys-Arg; 10) Trp-Arg-Tyr-Arg-Cys; 11) Arg-Cys-Trp-Trp-Trp, 12) Arg-Cys-Cys-Tyr-Trp; 13) Tyr-Trp-Cys-Tyr-Trp; and 14) Trp-Lys-His-Phe-Gln. The isolated peptide can further comprise a Met as the first N-terminal amino acid residue to facilitate recombinant production.

In still another specific embodiment, the present invention encompasses an isolated peptide for inhibiting a tyrosine recombinase, which peptide has the following formula:

(Xaa1-Xaa2-Xaa3-Xaa4-Xaa5-Xaa6)n

wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, Xaa6 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10. Preferably, Xaa1 is Trp, Arg or Tyr; Xaa2 is Trp, Lys, Arg or Cys; Xaa3 is Ala, His, Val, Tip, Arg, Cys or Tyr; Xaa4 is Trp, Cys, Tyr Phe or Arg; Xaa5 is Gln, Pro, Cys, Arg or Trp; and Xaa6 is Tyr, Arg, Phe or Trp. Exemplary peptides of this group include: 1) Trp-Lys-Ala-Tyr-Gln-Tyr; 2) Trp-Lys-His-Tyr-Pro-Tyr; 3) Trp-Lys-His-Tyr-Gln-Tyr; 4) Trp-Lys-Val-Tyr-Pro-Tyr; 5) Trp-Lys-Val-Tyr-Gln-Tyr; 6) Trp-Lys-Ala-Tyr-Pro-Tyr; 7) Trp-Arg-Arg-Trp-Cys-Arg; 8) Trp-Arg-Trp-Tyr-Cys-Arg; 9) Trp-Arg-Arg-Cys-Arg-Trp 10) Trp-Arg-Tyr-Arg-Cys-Arg; 11) Arg-Cys-Trp-Trp-Trp-Trp; 12) Arg-Cys-Cys-Tyr-Trp-Trp; 13) Tyr-Trp-Cys-Tyr-Trp-Trp; 14) Trp-Lys-His-Phe-Gln-Tyr; and 15) Trp-Lys-His-Tyr-Gln-Phe. The isolated peptide can further comprise a Met as the first N-terminal amino acid residue to facilitate recombinant production.

In yet another specific embodiment, the present invention encompasses the following isolated peptide for inhibiting a tyrosine recombinase: 1) Met-Trp-Lys-His-Tyr-Gln-Tyr; 2) Trp-Lys-His-Tyr-Gln-Tyr-Lys-Trp-Lys-His-Tyr-Gln-Tyr; and 3) Trp-Lys-His-Tyr-Gln-Tyr wherein each of the six amino acid residues is a D amino acid residue.

In yet another specific embodiment, the present invention encompasses an isolated peptide for inhibiting a tyrosine recombinase or a type I DNA topoisomerase, which peptide has the following formula:

(Xaa1-Xaa2-Xaa3-Xaa4)n

wherein each of Xaa1 and Xaa2 is an aromatic amino acid residue, Xaa3 is Ser, Cys or an aromatic amino acid residue, Xaa4 is an aromatic or a basic amino acid residue, wherein each of Xaa1, Xaa2, Xaa3, or Xaa4 can be a D or L amino acid residue and wherein n is an integer ranging from 1 to 10. Preferably, Xaa1 is Trp; Xaa2 is Trp; Xaa3 is Trp or Cys; and Xaa4 is Trp or Arg. Exemplary peptides of this group include: 1) Trp-Trp-Trp-Trp; 2) Trp-Trp-Trp-Arg; 3) Trp-Trp-Cys-Trp; and 4) Trp-Trp-Cys-Arg. The isolated peptide can further comprise a Met as the first N-terminal amino acid residue to facilitate recombinant production.

In yet another specific embodiment, the present invention encompasses an isolated peptide for inhibiting a tyrosine recombinase or a type I DNA topoisomerase, which peptide has the following formula:

(Xaa1-Xaa2-Xaa3-Xaa4-Xaa5)n

wherein Xaa1 is a basic amino acid residue, each of Xaa2 and Xaa3 is an aromatic amino acid residue, Xaa4 is Ser, Cys or an aromatic amino acid residue, Xaa5 is an aromatic or a basic amino acid residue, wherein each of Xaa1, Xaa2, Xaa3, Xaa4 or Xaa5 can be a D or L amino acid residue and wherein n is an integer ranging from 1 to 10. Preferably, Xaa1 is Lys or Arg; Xaa2 is Trp; Xaa3 is Trp; Xaa4 is Trp or Cys; and Xaa5 is Trp or Arg. Exemplary peptides of this group include: 1) Lys-Trp-Trp-Trp-Trp; 2) Lys-Trp-Trp-Trp-Arg; 3) Lys-Trp-Trp-Cys-Trp; and 4) Lys-Trp-Trp-Cys-Arg. The isolated peptide can further comprise a Met as the first N-terminal amino acid residue to facilitate recombinant production.

In yet another specific embodiment, the present invention encompasses an isolated peptide for inhibiting a tyrosine recombinase or a type I DNA topoisomerase, which hexapeptide has the following formula:

(Xaa1-Xaa2-Xaa3-Xaa4-Xaa5-Xaa6)n

wherein Xaa1 is a basic amino acid residue, each of Xaa2 and Xaa3 is an aromatic amino acid residue, each of Xaa4 and Xaa6 is Ser, Cys or an aromatic amino acid residue, Xaa5 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10. Preferably, Xaa1 is Lys; Xaa2 is Trp; Xaa3 is Trp; Xaa4 is Trp or Cys; Xaa5 is Trp or Arg; and Xaa6 is Trp or Cys. Exemplary peptides of this group include: 1) Lys-Trp-Trp-Trp-Trp-Trp; 2) Lys-Trp-Trp-Trp-Arg-Trp; 3) Lys-Trp-Trp-Trp-Trp-Cys; 4) Lys-Trp-Trp-Cys-Trp-Trp; 5) Lys-Trp-Trp-Cys-Arg-Trp; and 6) Lys-Trp-Trp-Cys-Trp-Cys. The isolated peptide can further comprising a Met as the first N-terminal amino acid residue to facilitate recombinant production.

In yet another specific embodiment, the present invention encompasses the following isolated peptides for inhibiting a tyrosine recombinase or a type I DNA topoisomerase: 1) Met-Lys-Trp-Trp-Cys-Arg-Trp; 2) Arg-Cys-Trp-Trp-Trp-Trp; and 3) Trp-Cys-Trp-Trp-Trp-Trp.

In the above-described peptides, the integer n ranges from 1 to 10. Preferably, n ranges from 1 to 5. More preferably, n ranges from 1 to 2.

The above-described peptides, can also comprise, consists essentially of, or consists of, a detectable label, such as a chemical label, e.g., streptavidin and biotin, an enzymatic label, e.g., LacZ and alkaline phosphatase, an radioactive label, e.g., ³H, ¹⁴C, ³³⁵S, ³²P and ¹²⁵I, a fluorescent label, e.g., GFP, BFP and RFP, or a luminescent label, e.g., luciferase. Preferably, the isolated and labeled peptide is biotinylated or fluorescently labeled at a Cys or Lys residue.

The peptides can be made by any methods known in the art. The peptides can be produced by chemical synthesis, recombinant production, or a combination thereof. Preferably, the peptides are produced by chemical synthesis (see e.g., Fmoc Solid Phase Peptide Synthesis: A Practical Approach, Chan and White (Ed.), Oxford University Press, 2000; Peptide Synthesis Protocols, Vol. 35, Pennington and Dunn (Ed.), Humana Press, 1995; and Chemical Approaches to the Synthesis of Peptides and Proteins, Lloyd-Williams et al. (Ed.), CRC Press, Inc., 1997). Also preferably, the peptides are screened and produced using the methods described in the above Section A.

Combinations and kits comprising the above-described peptides, which are useful for inhibiting cell growth, are also provided. Such combinations and kits contain, in addition to the peptides, other items such as packaging materials or usage instructions, etc.

D. Inhibition and Treatment Methods

In still another aspect, the present invention encompasses a method for inhibiting cell growth in a subject, which method comprises administering to a subject, to which such inhibition is desirable, an effective amount of an inhibitor of a site-specific DNA recombinase or a type I DNA topoisomerase, whereby cell growth is inhibited.

Any subject can be treated by the present method. Preferably, the subject being treated is a mammal. More preferably, the mammal being treated is a human.

The inhibitor of a site-specific DNA recombinase or a type I DNA topoisomerase can be administered alone, but is preferably administered with a pharmaceutically acceptable carrier or excipient.

Any site-specific DNA recombinase or type I DNA topoisomerase can be the therapeutic target. Preferably, the site-specific DNA recombinase to be inhibited is a tyrosine recombinase. Also preferably, the site-specific DNA recombinases or type I DNA topoisomerases inhibitor used in the treatment has the following formulas:

1) a peptide having the following formula:

(Xaa1-Xaa2-Xaa3-Xaa4)n

wherein each of Xaa1 and Xaa2 is an aromatic amino acid residue, Xaa3 is Ser, Cys or an aromatic amino acid residue, Xaa4 is an aromatic or a basic amino acid residue, wherein each of Xaa1, Xaa2, Xaa3, or Xaa4 can be a D or L amino acid residue and wherein n is an integer ranging from 1 to 10;

2) a peptide having the following formula:

(Xaa1-Xaa2-Xaa3-Xaa4-Xaa5)n

wherein Xaa1 is a basic amino acid residue, each of Xaa2 and Xaa3 is an aromatic amino acid residue, Xaa4 is Ser, Cys or an aromatic amino acid residue, Xaa5 is an aromatic or a basic amino acid residue, wherein each of Xaa1, Xaa2, Xaa3, Xaa4 or Xaa5 can be a D or L amino acid residue and wherein n is an integer ranging from 1 to 10; or

3) a peptide having the following formula:

(Xaa1-Xaa2-Xaa3-Xaa4-Xaa5-Xaa6)n

wherein Xaa1 is a basic amino acid residue, each of Xaa2 and Xaa3 is an aromatic amino acid residue, each of Xaa4 and Xaa6 is Ser, Cys or an aromatic amino acid residue, Xaa5 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

Other site-specific DNA recombinases or type I DNA topoisomerases inhibitors described in the above Section B can also be used.

In a specific embodiment, the subject being treated has or is suspected of having tumor or cancer. The neoplasms, tumors and cancers that can be treated include, but are not limited to, the neoplasm of adrenal gland, anus, auditory nerve, bile ducts, bladder, bone, brain, breast, bruccal, central nervous system, cervix, colon, ear, endometrium, esophagus, eye, eyelids, fallopian tube, gastrointestinal tract, head and neck, heart, kidney, larynx, liver, lung, mandible, mandibular condyle, maxilla, mouth, nasopharynx, nose, oral cavity, ovary, pancreas, parotid gland, penis, pinna, pituitary, prostate gland, rectum, retina, salivary glands, skin, small intestine, spinal cord, stomach, testes, thyroid, tonsil, urethra, uterus, vagina, vestibulocochlear nerve and vulva neoplasm. The present method can be used alone or can be used in combination with other an anti-tumor or anticancer agent, e.g., anti-angiogenic agents, or treatment, e.g., chemo- or radiation-therapy.

In another specific embodiment, the subject being treated is or is suspected of being infected by a bacterium and the inhibitor used in the method inhibits Holliday junction intermediate resolution activity of a tyrosine recombinase. Any bacterial infection, including infection by eubacteria or archaebacteria, by gram positive or gram negative bacteria, by an enteric pathogenic bacterium, by a SALMONELLA, a SHIGELLA, a STAPHYLOCOCCUS, a STREPTOCOCCUS or a BACILLUS species, or by E. coli., can be treated by the present method.

Any substance that inhibits Holliday junction intermediate resolution activity of a tyrosine recombinase can be used in the treatment. Preferably, the inhibitor has the following formulas:

1) a peptide having the following formula:

(Xaa1-Xaa2-Xaa3-Xaa4)n

wherein Xaa1 is an aromatic or a basic amino acid residue, Xaa2 is Ser, Cys, an aromatic or a basic amino acid residue, Xaa3 is any amino acid residue, Xaa4 is Ser, Cys, an aromatic or a basic amino acid residue, wherein each of Xaa1, Xaa2, Xaa3, or Xaa4 can be a D or L amino acid residue and wherein n is an integer ranging from 1 to 10;

2) a peptide having the following formula:

(Xaa1-Xaa2-Xaa3-Xaa4-Xaa5)n

wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, wherein each of Xaa1, Xaa2, Xaa3, Xaa4 or Xaa5 can be a D or L amino acid residue and wherein n is an integer ranging from 1 to 10; and

3) a peptide having the following formula:

(Xaa1-Xaa2-Xaa3-Xaa4-Xaa5-Xaa6)n

wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, Xaa6 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

The present method can be used alone or can be used in combinaiton with other antibiotics or other anti-bacterium treatments.

The formulation, dosage and route of administration of the cell growth inhibitors, e.g., the peptide inhibitors described above and in Section B, can be determined according to the methods known in the art (see e.g., Remington: The Science and Practice of Pharmacy, Alfonso R. Gennaro (Editor) Mack Publishing Company, April 1997; Therapeutic Peptides and Proteins: Formulation, Processing, and Delivery Systems, Banga, 1999; and Pharmaceutical Formulation Development of Peptides and Proteins, Hovgaard and Frkjr (Ed.), Taylor & Francis, Inc., 2000). The cell growth inhibitors can be formulated for oral, rectal, topical, inhalational, buccal (e.g., sublingual), parenteral (e.g., subcutaneous, intramuscular, intradermal, or intravenous), transdermal administration or any other suitable route of administration. The most suitable route in any given case will depend on the nature and severity of the condition being treated and on the nature of the particular active cell growth inhibitor which is being used.

This invention will be more completely described by means of the following examples, which are to be considered illustrative and not imitative.

### EXAMPLES

E. Dissection of Bacteriophage λ Site-Specific Recombination Using Synthetic Peptide Combinatorial Libraries

In order to add to the repertoire of available tools of analyzing site-specific recombination, we have investigated a different class of reaction inhibitors, namely hexapeptides, which would help us dissect the site-specific recombination pathways. Our rationale was based on two assumptions: first, Int probably uses different, though perhaps overlapping, protein surfaces for the cleavage versus the ligation steps, and thus we should be able to find distinct inhibitors for each of these reactions. Second, certain reaction intermediates have unique conformations and might be stabilized by compounds which interact specifically with these intermediates; an example of such an intermediate is the Holliday junction formed after the first round of cleavage and ligation steps (FIG. 1). In this work, we describe the identification of peptides which block recombination early in the pathway and peptides which trap the Holliday junction intermediate. The identification of peptides which trap covalent Int-DNA complexes are described in the following Section G.

Our strategy was to use a combinatorial approach to look for peptides which affect Int action among systematically arranged mixtures of hexapeptides having either one or two positions defined (Houghten et al., 1991; Pinilla et al., 1992). Synthetic peptide combinatorial libraries (SPCLs) were more attractive than phage display libraries because we anticipated that the inhibitors would have to diffuse into small pockets between two Integrase monomers or between the enzyme active site and its DNA substrate. Moreover, the diversity of SPCLs is quite high (for each position of the hexameric peptide, we screened 20 mixtures each containing nearly 2.5×10⁶ different peptides), thus increasing the likelihood of success.

We have found specific peptides that block DNA cleavage with an IC₅₀ value as low as 0.02 μM and distinct specific peptides which stabilize the Holliday junction intermediate of Int-mediated recombination with an IC₅₀ value as low as 0.2-0.4 μM. Our results suggest that this approach should be widely applicable to the dissection of any biochemical reaction into substituent steps, whether the intermediates are known a priori or not.

Results

In addition to integrative and excisive recombination, Int also carries out unidirectional recombination reactions in which the products are the same as the substrates. One of these is the bent-L pathway and has been reconstituted in vitro (Segall and Nash, 1996). There were several advantages to using this recombination pathway in the screen for peptide inhibitors: 1) the bent-L pathway has fewer requirements than integration or excision (only Int, IHF and one type of substrate are necessary) without any sacrifice in recombination efficiency, and 2) many higher-order intermediates in this pathway have been described, including synaptic intermediates (Segall, 1998). In contrast, the synaptic intermediates of the excision and integration pathways are too transient to isolate. Few catalytic intermediates accumulate in any pathway of λ site-specific recombination: in the bent-L pathway specifically, fewer than 4% of substrates accumulate as covalent protein-DNA complexes (CPDs), and fewer than 2% of substrates accumulate as HJs (see below). Nevertheless, we supposed that inhibitors effective against the bent-L pathway may be useful for studying all of the Int-mediated pathways due to common catalytic steps.

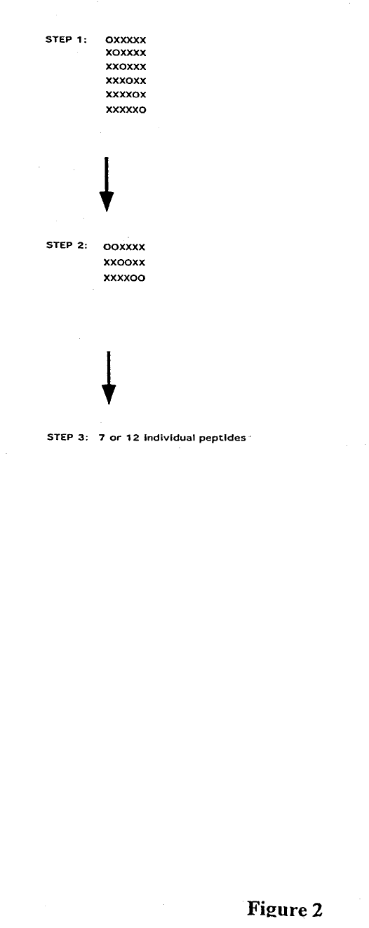

Peptide library deconvolution protocol. We have used the positional scanning strategy for peptide library deconvolution (Pinilla et al., 1992; reviewed in Houghten et al., 1999). The first step consisted of the screening of six different sets, each containing 20 peptide mixtures in which one position was fixed (represented by each of the 20 amino acids) and the remaining 5 positions were mixtures of 19 amino acids (all except cysteine; FIG. 2 step 1). We thus tested a total of 120 mixtures in the first step. Recombination reactions containing two concentrations of each peptide library were screened for the appropriate phenotype, and mixtures which conferred the strongest phenotype were further tested in dose response assays to identify the most potent mixtures. We did not proceed directly to synthesizing specific peptides at this step. Because we had identified between 3 and 6 candidate amino acids at each position, synthesizing peptides containing all possible combinations of these amino acids would have been both very expensive and impractical. Instead, in the second step of the screen we sampled the same diversity of compounds in a different way: we used libraries of peptides which contained defined amino acids at two positions and mixtures of 19 amino acids at the remaining 4 positions, which are known as dual-defined libraries (Appel et al., 1996, Dooley et al., 1997). While the complete library of compounds would be represented by 1200 mixtures (3 sets of 400 mixtures), we only tested the subset of mixtures in which the defined amino acids present in the most active mixtures from step 1 were combined in pairs of defined neighboring positions (FIG. 2 step 2). These mixtures have a lower complexity of peptides (approximately 130,000 peptides/mixture) and therefore each peptide is present at higher concentration, allowing for better discrimination between peptides. In addition, these mixtures begin to highlight combinations of neighboring amino acid residues that are most active together. Finally, this information was used to select the amino acid pairs from the most active dual-defined position mixtures, and to synthesize a number of specific peptides that were individually added to recombination reactions and tested in dose response assays to obtain the IC₅₀ value (FIG. 2 step 3).

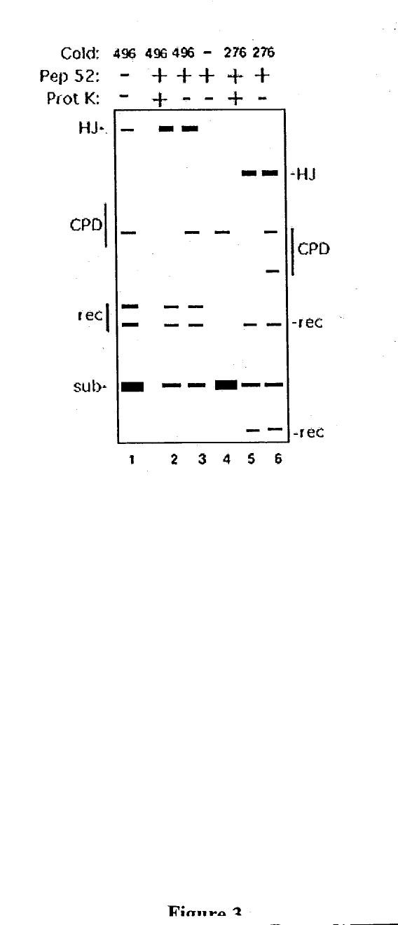

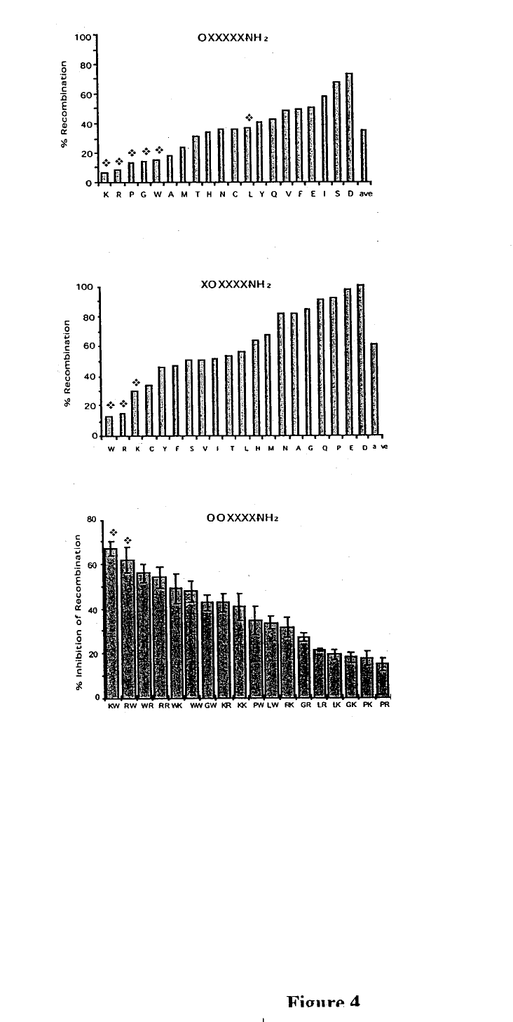

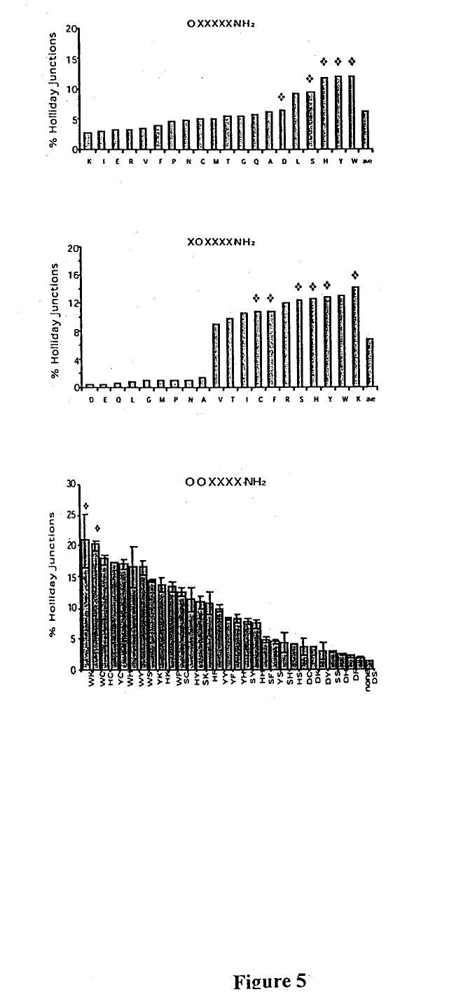

Int was pre-incubated with each of 120 peptide mixtures of the single-position defined PS-SPCLs at 2 different concentrations and added to recombination reactions. A low level of CPD complexes and HJs can be seen in the untreated reactions. The CPDs are sensitive to proteinase K and contain Int attached either to the substrates or to the recombinant, products (FIG. 3). In contrast, the HJs are proteinase-K and SDS-resistant but migrate differently depending on the size of both labeled and unlabeled substrates in the reaction (FIG. 3). Depending on the mixture added to each reaction, recombination efficiency was decreased more or less drastically, and HJs accumulated to different extents. Based on these results, we identified mixtures with amino acids at each position of the hexapeptide that led to the greatest repression of recombination (FIG. 4) or the greatest accumulation of HJs (FIG. 5). Dose response titrations were performed with mixtures showing the highest activity in order to determine the most potent mixture with respect to each phenotype (data not shown). In addition, some mixtures caused an increase in the CPDs without concomitant increase in Holliday junctions. We reasoned that these mixtures contain peptides which may interfere with Int-mediated ligation while allowing DNA cleavage.

The amino acids identified as the most potent from the single fixed position mixtures were paired and the resulting dual defined position mixtures were tested as above (FIG. 2 step 2). The dual defined position mixtures were ranked according to their potency at inhibiting recombination or at accumulating HJs (examples are shown in FIG. 4 and FIG. 5), and dose response titrations were performed to identify the most active mixtures (data not shown). This intermediate step tested the most active neighboring pairs of amino acids and allowed us to reduce the number of individual peptides we had to synthesize.

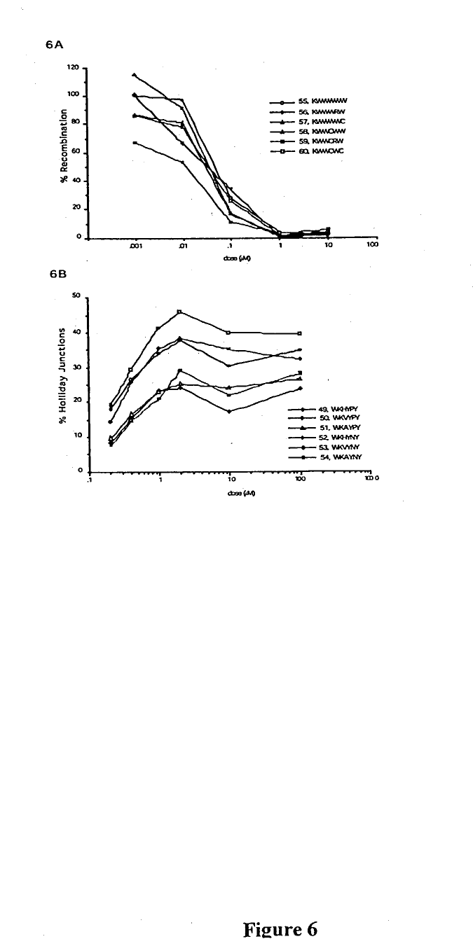

Based on the ranking of the selected dual defined mixtures, individual peptides were synthesized (FIG. 2 step 3) and their effect on recombination reactions was tested (peptide sequences are shown in FIG. 6). Dose response curves showed that all the resulting peptides affected recombination, although some were more potent than others. Peptides 59 and 56 were the most potent at inhibiting recombination (FIG. 6A), while peptides 54, 52 and 49 were the most potent at causing the accumulation of HJs (FIG. 6B). A second set of 6 peptides, containing Cys instead of Lys at the second position but otherwise identical to peptides 49-54, was tested for accumulation of HJs. This set was significantly less potent (at least 10 fold higher IC₅₀; data not shown). Henceforth we focus on peptides 52 and 59. Note that the top-ranked specific peptide sequence did not necessarily follow the ranking of the amino acid pairs predicted in the screening of the dual defined position libraries.

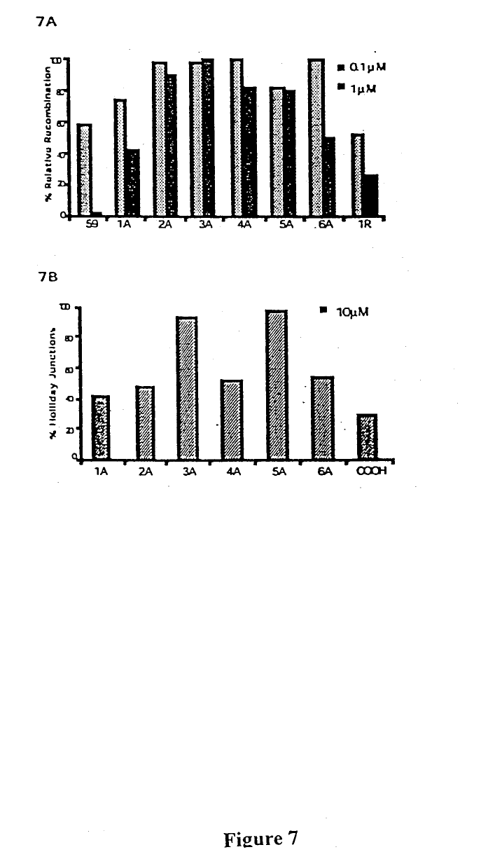

Characterization of individual peptides. In order to test the importance of specific amino acids in the final hexapeptides, peptides substituted with alanine at each position were synthesized and tested. The results agreed with data obtained during the library deconvolution process: positions in which alanine could be substituted without significant loss of potency coincided with positions in which a higher number of amino acids were effective at eliciting the phenotype (FIG. 7 and data not shown). Although conservative substitutions were well supported (e.g., arginine was nearly as effective as lysine at position 1 in peptide 59; FIG. 7A), each position in peptide 59 contributed significantly to the overall potency of the peptide at inhibiting recombination. In contrast, positions 3 and 5 in peptide 52 could be substituted with alanine with little or no effect on the peptide's activity (FIG. 7B), in agreement with our data that peptides which differed only at these positions had similar activities in dose response assays (FIG. 6B). In addition, in the case of peptides 49 through 56, we found that the C-terminal amide group is an important constituent for the activity of the peptides; substituting this with a carboxyl group results in about 50% decrease in activity (FIG. 7B). Similar observations have been made for peptides with other biological activities, but is not the case for peptide 59 and related peptides (data not shown).

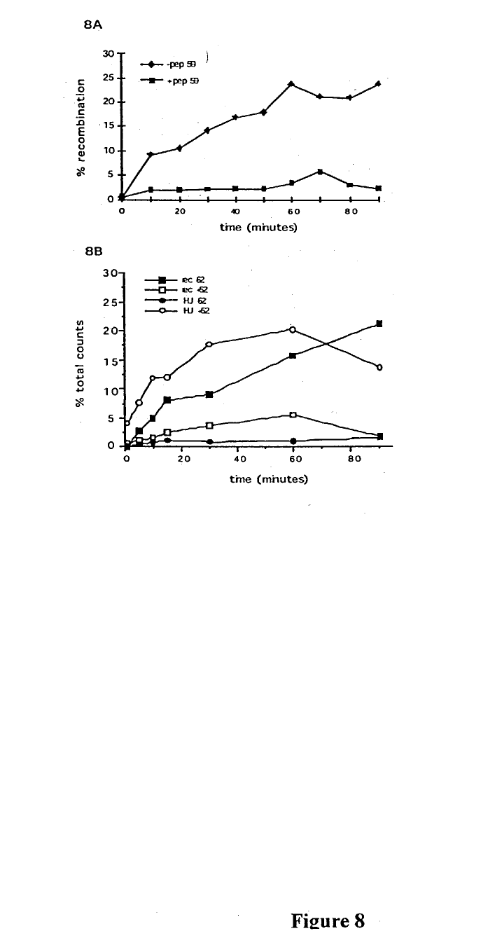

Timecourses were performed in order to determine the effect of peptides 59 and 52 at different stages of recombination. Peptide 59 inhibits recombination early and recombination levels do not recover at later time points, suggesting that the association of the peptide with its target(s) in the recombination complex is stable (FIG. 8A). This has been confirmed with dilution assays (data not shown). Based on this and other data (see following Section F), peptide 59 appears to inhibit Int cleavage of DNA.

Peptide 52 decreases recombination but does not inhibit it completely even at 100 μM, the highest peptide concentration tested (FIG. 8B; data not shown). However, reactions treated with the peptide accumulate HJs as the reaction proceeds. Peptide 52 probably does not inhibit the first strand cleavage event, since this would preclude accumulation of HJs. If the peptide simply inhibited the second strand cleavage which resolves the HJ intermediates, we would expect that a high proportion of the resulting HJs would be reversed to substrates (this is what occurs when the second strand cleavage event is blocked by a phosphorothiolate substitution; Kitts and Nash, 1987). This is not the case, however, suggesting that the peptide may bind and stabilize the Holliday junction intermediate. This model has been supported by subsequent experiments (see following Section G).

We have used the BLAST algorithm (Altschul et al., 1997) to look for any structural similarities between the peptides and their target enzymes or with any known protein. The only match we have found is between the last 5 residues of peptide 56 (KWWWRW) and the HIV1 envelope glycoprotein. We conclude that the sequence of the peptides clearly could not have been predicted or derived from the structure of either Int or related proteins.

Discussion

Using a positional scanning approach to deconvolute synthetic peptide combinatorial libraries, we have identified 2 distinct types of peptides which affect different steps of the Int-mediated bent-L pathway of λ site-specific recombination. One set of peptides, represented by peptide 59 (KWWCRW, blocks recombination early in the pathway, while the second set of peptides, represented by peptide 52 (WKHYNY) leads to the accumulation of Holliday junctions and does not inhibit recombination completely even at 100 μM. The two families of peptides have different sequences, as expected for molecules that interact with different targets or with distinct surfaces of the same target. They also have distinct profiles in the alanine scanning experiments: substitution with alanine of any single residue in peptide 59 (KWWCRW) significantly or completely abates the peptide's activity, while the 3rd and 5th residues in peptide 52 (WKHYNY) can be substituted with alanine without diminishing its activity. The two peptide families also share some attributes: each has at least one positively charged residue, and 3 out of the 6 amino acids are aromatic, leaving open the possibility that these peptides may intercalate into or otherwise interact with DNA. In addition, since both peptide 59 and peptide 52 are hydrophobic, they may interface between the proteins and DNA substrates within recombination complexes. Neither of them, however, interferes with Int-mediated assembly of recombination intermediates (see following Section F). Interestingly, the third phenotype—accumulation of covalent protein-DNA complexes—identified a set of amino acids which included many more charged and fewer hydrophobic residues. Neither peptide 52 nor peptide 59 resemble any portion of Int or of the accessory factors involved in Int-mediated recombination.

Both peptides 52 and 59 affect the other pathways of λ site-specific recombination in a similar manner, although with different potencies (see following Sections F and G). These peptides have provided us with important new tools for dissecting the various stages of site-specific recombination, and for analyzing the structure and protein-DNA interactions within intermediates which have not been well-characterized. For example, the accumulation of high levels of the HJ intermediate has not been achieved either with mutant Int proteins or with DNA modifications.

The bent-L recombination pathway offered several advantages as a reaction to validate the usefulness of the mixture-based combinatorial libraries for dissecting a biochemical pathway. The reaction progresses through a series of defined higher order protein-DNA intermediates (Segall, 1998). While catalytic intermediates in the pathway were not similarly well characterized, the effect of specific peptides on these intermediates could be tested subsequently (see following Sections F and G). The assay is sufficiently reproducible so that changes of 10% or less in extents of recombination or intermediate formation were easily detectable. Measuring intermediates was easier because so few accumulate in the absence of peptide inhibitors.

The power of the deconvolution approach lies in the ability to identify a few potent compounds among mixtures containing millions of different compounds with little or no effect on the reaction (reviewed by Houghten et al., 1999). Although in the first step of deconvolution (FIG. 2) the concentration of any individual peptide is very low (about 1.25 nM in our case, because each single-position defined library is present at a final concentration of 1 mg/ml in the reaction), each mixture contains many members that are closely related (1 or 2 amino acids away) and have some activity in the assay. These related peptides, though they may be less potent, help increase the effective concentration of the most potent peptide (for discussion, see Houghten et al., 1999). To illustrate, since peptide 52 (WKHYNY) has an IC₅₀ of 200 nM, roughly 160 peptides should exhibit some related behavior in order to increase the effective concentration of this peptide from 1.25 nM to 200 nM (the IC₅₀) in step 1 of the deconvolution process. FIG. 6B shows that 5 other peptides have IC₅₀ values within 3 fold of peptide 59. Since each tyrosine can be substituted with phenylalanine with less than 2-fold loss of potency (data not shown), 12 more peptides have significant activity in the assay. FIG. 5 shows that two amino acids could be substituted at position 1 and five at position 2, bringing the number of peptides that have a phenotype similar to that of peptide 59 from 1 to 270. In step 2 of the deconvolution process (FIG. 2), the concentration of any individual peptide is higher since the complexity of the library is lower. Nevertheless, the same logic applies: the effective concentration of the most potent peptide is increased due to the activity of related peptides in each mixture.

Combinatorial methods such as the SELEX protocol and phage display libraries have been extremely powerful in identifying enzyme inhibitors, nucleic acid binding sites, or protein ligands (Tuerk and Gold, 1990; Lowman, 1997; articles in Methods in Enzymology vol. 267). Nevertheless, it is unlikely that either the SELEX or phage display approaches would have identified nucleic acids or peptides with the phenotypes described here. Both of these approaches select compounds based on their ability to bind a component of the reaction and depend on the ability of the assay to detect such binding. At a concentration of 5 μM and above, peptide 59 does shift the mobility of double-stranded DNA in our reactions, which contain at least 50 ng salmon sperm DNA (see following Section F). However, screening or selecting peptides based on this phenotype would probably have been unsuccesful in leading us to peptide 59, since the initial concentration (as well as the effective concentration; see above) of this peptide in the single fixed position SPCLs is well below the concentration at which DNA binding can be seen in a mobility shift assay. Moreover, we would not have been able to identify peptide 52 based on binding interactions either with DNA or with Int. Extensive order-of-addition experiments and titration experiments have shown that neither Int alone nor the DNA alone are the target of the peptide (see following Section F). Rather, our data suggest the possibility that both peptides interact with an Int-DNA complex, although they have different targets within that complex (see following Sections F and G). More importantly, peptides displayed on phage may not have had adequate access to protein-DNA or protein-protein interfaces within the recombination complexes.

SPCLs have had only limited use in studying enzymes which act on DNA. Plasterk and colleagues (Puras Lutzke et al., 1995) have deconvoluted peptide libraries based on inhibition of HIV integrase DNA cleavage activity, and have secondarily characterized the effect of the resulting peptides on other steps in the pathway. They did not, however, deconvolute libraries based on the accumulation of intermediates. We suggest that the potential of these libraries as tools has been underappreciated.

In summary, we believe that the mixture-based library deconvolution approach is applicable to any biochemical pathway which has been reconstituted in vitro either in a pure or semi-pure system, and may work equally well in cell extracts. Intermediates need not have been identified a priori, as long as the assay used in the deconvolution process is reproducible and has the potential to detect suspected intermediates. Finally, while the deconvolution of mixture-based libraries could be automatable, the approach is not so onerous as to prevent its use with commonly available molecular and biochemical assays.

Materials and Methods

DNA substrates and proteins. Substrates were synthesized by PCR using plasmid templates with cloned attL, attL tenP′1, attR or attB sites as described (Segall et al., 1994). Substrates were 5′ end-labeled with γ-³²P-ATP (New England Nuclear) using T4 polynucleotide kinase (New England Biolabs). Purified Int was the generous gift of C. Robertson and H. Nash (NIH), and of J. Hartley (Life Technologies Inc.). Purified IHF was the generous gift of S.-W. Yang and H. Nash (NIH), while purified Xis was the generous gift of C. Robertson and H. Nash (NIH).

Recombination Assays. Recombination assays were performed as described (Segall, 1998). Briefly, reactions were performed in a total volume of 10 μl and typically contained 1 nM radiolabeled att site as specified, 4 nM unlabeled att site, 50 ng salmon sperm DNA as nonspecific competitor, 44 mM Tris-Cl (pH 8.0), 60 mM KCl, 0.05 mg/ml bovine serum albumin, 7-11 mM Tris borate (pH 8.9), 5 mM spermidine, 1.3 mM EDTA, and 14.6% v/v glycerol. Int and IHF were present at 55 nM and 35 nM final concentrations respectively. During screening, peptide libraries were incubated with Int on ice for 20 minutes (in the same buffer), and the mix was then added to the rest of the recombination reaction. Final concentrations of peptides are specified for each experiment. Reactions were incubated for 60-90 minutes at 30° C. or 37° C., were stopped with 0.2× volume of 2% SDS, layered onto 5% polyacrylamide Tris/SDS gels, and electrophoresed in 1× Tris Tricine SDS buffer at 100 mA (Segall, 1998). Dried gels were visualized and quantitated using a Molecular Dynamics PhosphorImager.

Peptide libraries. Peptide libraries were synthesized at Torrey Pines Institute for Molecular Studies using TBOC-protected L amino acids as described (Pinilla et al., 1992). Because some peptide libraries contain up to 0.5% NaF, we tested the effect of NaF on recombination and found that recombination is unaffected by up to 1% NaF (data not shown).The dual-defined position libraries were dissolved in DMSO; therefore, “untreated” reactions contained the appropriate final concentration of DMSO without peptides. Peptides of specific sequence were synthesized either at Torrey Pines Institute for Molecular Studies or at Sigma-Genosys Inc. (the latter were synthesized using FMOC-protected L-amino acids).

F. Peptide Inhibitors of DNA Cleavage by Tyrosine Recombinases and Topoisomerases

We have identified hexapeptides that efficiently block recombination at an early step (See above Section A)). In this Section, we describe the activities of two of these peptides, KWWCRW and KWWWRW, and show that they block DNA cleavage catalyzed by bacteriophage λ Integrase. In the following Section G, we describe another set of peptides that trap the Holliday junction intermediate of Int-mediated recombination.

Tyrosine recombinases conserve the energy of the cleavage event and use it for the ligation event. The same strategy is employed by DNA topoisomerases, which are divided into 2 major classes (Wang, 1985). The type I enzymes cut DNA one strand at a time, whereas the type II enzymes cut both DNA strands at once. In turn, the type I enzymes are themselves subdivided into 2 subclasses, IA and IB, based on whether a free 3′ OH or a 5′ OH is generated after nucleophillic attack. Because the tyrosine recombinases have a related mechanism and structural similarity to the eukaryotic type IB topoisomerases (Cheng et al., 1998, Redinbo et al., 1998, Stewart et al., 1998; Redinbo et al., 1999), the inhibitory activity of the peptides was tested on the smallest and best studied of these enzymes, the vaccinia virus topoisomerase. For comparison, we also tested the inhibition by peptides of type IA and type II topoisomerases and of several restriction enzymes. We show that the peptides inhibit DNA cleavage with an effectiveness more or less related to the evolutionary similarity of these enzymes to each other: the peptides inhibit bacteriophage λ Integrase best, vaccinia topoisomerase with somewhat lower potency, are less potent against the E. coli type IA topoisomerase I, and are least potent against the type II T4 topoisomerase and restriction enzymes.

Results

Peptide inhibition of λ Integrase

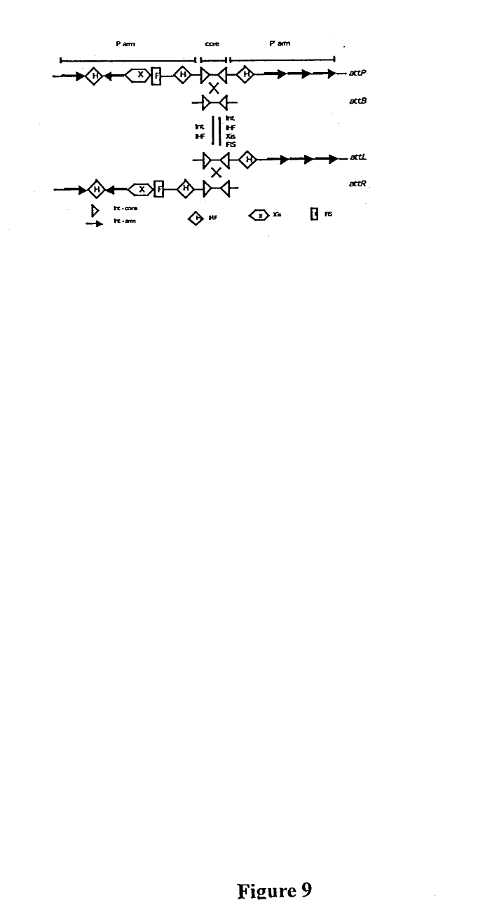

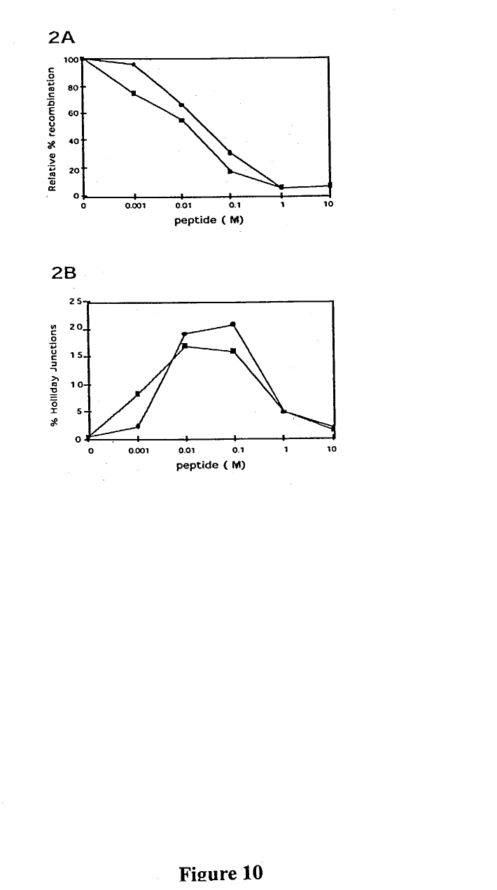

Several hexameric peptides which inhibit the Int-mediated bent-L recombination pathway were identified by screening synthetic peptide combinatorial libraries using a positional scanning strategy (see the above Section E; Pinilla et al., 1998). Two related peptides, KWWCRW (peptide 59) and KWWWRW (peptide 56), showed the strongest phenotype. The effect of KWWCRW on the bent-L reaction is shown in FIG. 10. At 10 μM peptide, recombination was inhibited completely without accumulation of intermediates. The concentration of peptide that inhibited recombination 50% (IC₅₀) was less than 0.1 μM (FIG. 10A). At intermediate peptide concentrations (1 μM-0.01 μM), the proteinase K-resistant Holliday junction accumulated as recombination gradually increased. At concentrations below 0.01 μM, recombination levels approached that of untreated reactions (FIG. 10A). The accumulation of Holliday junctions was maximal at peptide concentrations which did not completely inhibit recombination (1 μM-0.1 μM; FIG. 10A versus FIG. 10B). The peptides did not increase the level of protein-DNA covalent intermediates (CPDs; see FIG. 9), showing that the ligation event was unaffected. In fact, peptide concentrations that blocked recombination also inhibited formation of these CPDs. These data suggest that the peptides inhibit Int-mediated DNA cleavage, and that the interaction of more than one peptide with the protein and/or DNA components of the system is necessary to completely inhibit recombination. Because each complete round of recombination involves 4 DNA cleavage events, a suboptimal number of peptides inhibits some but not all DNA cleavages and Holliday junctions accumulate.

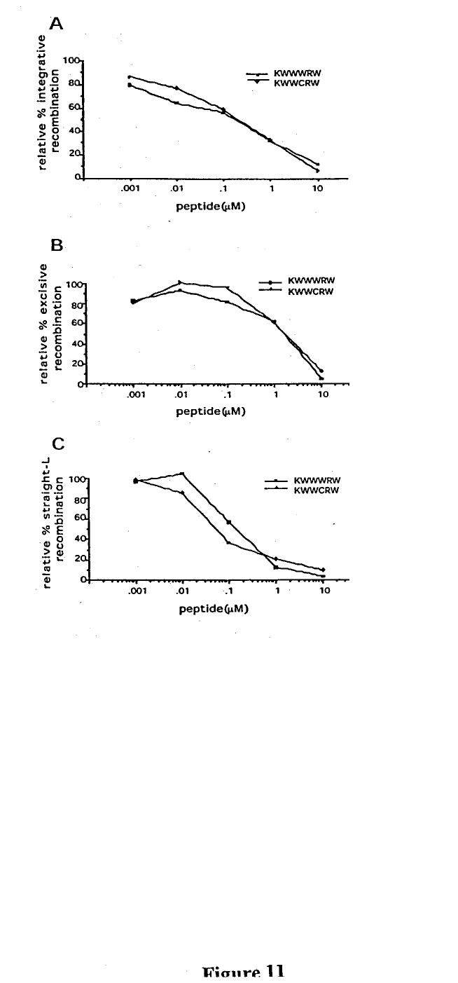

During peptide library deconvolution, we used the bent-L recombination pathway because it is efficient, it involves only Int and IHF, and it uses linear substrates (Table 1). We next tested whether peptides KWWCRW and KWWWRW inhibit the integrative, excisive and straight-L recombination reactions. Although all pathways were affected, the potency of the peptides differed in each pathway (FIG. 11). KWWCRW was most effective in bent-L recombination (IC₅₀=0.02 μM), less effective in straight-L recombination (IC₅₀=0.06 μM) and integration (IC₅₀=0.2 μM), and least effective in excision (IC₅₀=1.1 μM). KWWWRW had a very similar potency profile. Although Int is the agent of DNA cleavage in all 4 pathways, Int carries out cleavage within intermediate complexes having distinct, pathway-specific conformations (Segall and Nash, 1996). Because neither IHF nor Xis proteins are involved in the straight-L pathway, either DNA and/or Int must be the target of the peptides. However, order-of-addition experiments and titration experiments have not identified Int alone or DNA alone as the target (data not shown), suggesting instead that an Int-DNA complex is the target. Our data indicate either that Int interacts with its substrates in a somewhat different way in each recombination pathway, thus presenting a somewhat different target for the peptide, or that the target is the same in each pathway but the abundance of this target complex differs in each pathway (see below).

TABLE 1
Summary of the 4 pathways of bacteriophage λ site-specific recombination.

| Pathway: | Integration | Excision | Bent-L | Straight-L |
|---|---|---|---|---|
| att substrates | attP, attB | attL, attR | attL (tenP'1)^{a} | attL |
| Int requirement | Y | Y | Y | Y |
| Bending protein requirement | IHF | IHF > HU, HMG1, 2 | IHF | inhibitory |
| Xis requirement | inhibitory | Y | N | N |
| supercoiling requirement | Y | N | N | N |
| Efficiency^{b} | high | high | high | low |

^{a}The bent-L pathway in vivo works equally well with wild type attL or attL tenP'1 substrates. However, the pathway works only with attL tenP'1 substrates in vitro (Segall and Nash, 1996).
^{b}High efficiency denotes >25% conversion of substrates to products. Low efficiency denotes <5% conversion of substrates to products.

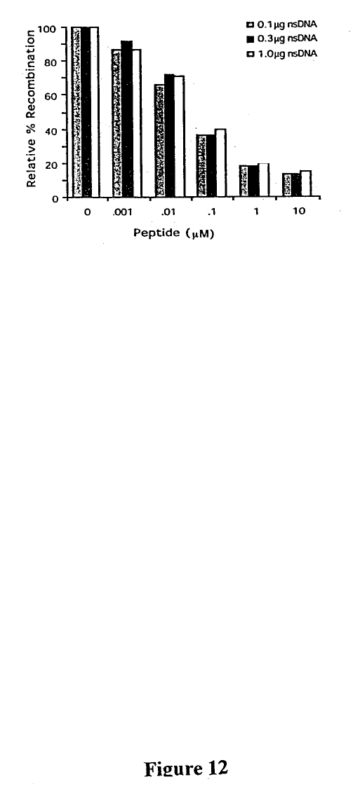

Do peptides KWWCRW and KWWWRW inhibit recombination by interfering with the formation of higher order complexes? The formation of intermediates in the bent-L pathway depends on Int contacting two different types of sites, the higher affinity arm sites and the lower affinity core sites flanking the loci of DNA cleavage and strand exchange (FIG. 9). In an electrophoretic mobility shift assay, we found that both peptides interfered slightly with contacts between Int and its arm binding sites. To determine the effect of KWWCRW on interactions of Int with its core binding sites, we assembled the recombination complexes, known as intasomes or unimolecular complexes (UMC), on an attL variant substrate with 4 mutations in the IHF binding site, collectively known as QH′. These mutations prevent the specific binding of IHF to the QH′ sequence (Gardner and Nash, 1986), but still allow IHF to bind and bend DNA nonspecifically (Segall et al., 1994). In this situation, the appropriate complex can only be formed in the presence of Int and only when IHF binds in a “pseudo sequence specific” manner and bends DNA at the appropriate site; this situation demands more stable Int-core interactions than are necessary when IHF binds and bends the attL site in a sequence-specific fashion (Segall et al., 1994). The peptides did not interfere with formation of the Int/IHF/attL-QH′ complex, despite the peptide's effect on arm binding of Int. This suggested that the overall stability of the intasome suppressed the negative effect of the peptides on arm binding by Int. We next tested the assembly of bent-L pathway intermediates. At 10 μM peptide, all of the labeled DNA was shifted into the well. However, at lower peptide concentrations that still inhibited recombination (0.1-1 μM), intermediates were assembled normally. In fact, one of the intermediates, the bimolecular complex (BMC), accumulates substantially in the presence of the peptide (see also FIG. 12). When this intermediate was analyzed on a second, SDS-containing gel, it was found to contain Holliday junctions (data not shown). This agrees perfectly with our observations that suboptimal concentrations of peptide lead to accumulation of Holliday junctions (FIG. 10). Both KWWCRW and KWWWRW appear to bind to DNA, although the reactions contain 100 ng of salmon sperm DNA in addition to the att substrates. The peptide shifts att site DNA even in the complete absence of Int (data not shown), confirming that KWWCRW interacts with DNA in a concentration-dependent fashion and in a manner that affects the mobility of the DNA much more drastically than expected for the size of the peptides.

We examined whether the inhibitory properties of the peptide were correlated with its DNA binding by testing the effect of increasing concentrations of salmon sperm DNA on the mobility and assembly of intermediates and on recombination. The results showed that the presence of 0.3 μg salmon sperm DNA concentration reversed the effect of 10 μM peptide concentration on the mobility of att intermediates. However, the presence of 0.3-1 μg salmon sperm DNA did not reduce the peptide's ability to inhibit recombination (FIG. 12). We interpret these results to mean that the peptides either exhibit sequence-specific DNA binding or display a high affinity for some conformational feature specific to recombination intermediates.

Int, like its relative tyrosine recombinases, makes transient covalent protein-DNA complexes (CPDs) during the cleavage stage of the recombination reaction. While most of these complexes proceed through strand exchange and ligation, a small percentage of them do not and can be visualized on SDS-containing gels as proteinase K-sensitive species (e.g., FIG. 10 and the above Section E). Since these complexes are the product of DNA cleavage by Int prior to strand exchange or ligation, we tested the effect of peptide KWWCRW on their formation. The peptide inhibited accumulation of both attL and attR CPDs by 65-75% (data not shown), supporting our model that KWWCRW blocks recombination by interfering with the cleavage step of the reaction.

In order to test the specificity of peptide inhibition, we determined whether peptide KWWCRW affects the activity of a relative of the Int recombinase, namely the bacteriophage P1 Cre protein. Indeed, the peptide inhibits Cre-mediated recombination between two lox site substrates (Cassell and Segall, unpublished results). Based on these results, we asked whether the peptides inhibit enzymes with similar mechanisms of action that are less closely related to Int.

Peptide Inhibition of Vaccinia Topoisomerase.

Vaccinia virus topoisomerase, a prototypal type IB enzyme, is structurally and mechanistically similar to the tyrosine recombinases (Cheng et al., 1998). The anti-Int peptides inhibit the DNA relaxation activity of vaccinia topoisomerase. The reaction mixtures contained the minimum amount of input topoisomerase that sufficed to relax the pUC19 DNA to completion in 5 minutes, as determined by end-point dilution in 2-fold increments (data not shown). Peptides KWWWRW and KWWCRW inhibited DNA relaxation in a concentration-dependent manner. Activity was abolished at 10-15 μM peptide and reduced by one-half at approximately 3-4 μM peptide (Table 2). Two other aromatic hexapeptides, WCHYNY and WKHYNY, had no effect on DNA relaxation by vaccinia topoisomerase at peptide concentrations up to 42 μM (data not shown). These latter two peptides appear to stabilize Holliday junctions but by a different mechanism than peptides KWWCRW or KWWWWRW see the above Section E).

TABLE 2
Summary of IC₅₀ values for KWWCRW

| Protein: | IC₅₀ (μM) |
|---|---|
| Integrase: | |
| Bent-L | 0.02 |
| Straight-L | 0.06 |
| Integration | 0.3 |
| Excision | 1.1 |
| Vaccinia topoisomerase (type Ib) | 0.5^{a} (3.5) |
| E. coli topoisomerase I (type Ia) | 8 |
| T4 topoisomerase (type II) | 40 |
| Hind III^{b} (AAGCTT) | 48 |
| Nde I (CATATG) | 37 |
| Pst I (CTGCAG) | 44 |
| Xba I (TCTAGA) | 37 |

^{a}IC₅₀ for DNA cleavage is given, with the IC₅₀ for plasmid relaxation in parentheses. In the plasmid relaxation assay, most of the plasmid DNA can be considered nonspecific competitor DNA; this “extra” DNA is absent in the DNA cleavage assay.
^{b}The sequence of the recognition sites for each restriction enzyme is given in parentheses

The catalytic cycle of vaccinia topoisomerase entails multiple steps: (i) noncovalent binding of enzyme to duplex DNA; (ii) scission of one strand with concomitant formation of a covalent DNA-(3′-phosphotyrosyl)-topoisomerase adduct; (iii) strand passage; and (iv) strand religation. Vaccinia topoisomerase displays stringent sequence specificity in DNA cleavage; it binds and forms a covalent adduct at sites containing the sequence 5′(C/T)CCTT↓ (Shuman and Prescott, 1990). This feature of the vaccinia enzyme facilitates analysis of the partial reactions using model substrates containing a single CCCTT cleavage site. “Suicide” substrates have been especially useful for studying the cleavage reaction (first transesterification) under single-turnover conditions. Covalent adduct formation is accompanied by spontaneous dissociation of the 3′ fragment of the cleaved strand from the topoisomerase-DNA complex, which leaves a 18-nucleotide single-strand tail on the noncleaved strand. With no readily available acceptor for religation, the topoisomerase is covalently trapped on the DNA. The single-turnover reaction is complete within 15 s at 37° C. The yield of covalent adduct is proportional to input topoisomerase when DNA is in excess and the reaction is near-quantitative at saturating enzyme. Peptide effects were evaluated at enzyme concentrations sufficient to cleave 60-70% of the input substrate in 10 s. Peptides KWWWRW and KWWCRW, which blocked DNA relaxation, were potent dose-dependent inhibitors of covalent adduct formation (99% inhibition at 1.6 to 1.8 μM; IC₅₀ at ˜0.5 μM; Table 2), whereas peptides WKHYNY and WCHYNY did not inhibit transesterification (data not shown). Inhibition of DNA cleavage by KWWWRW and KWWCRW as a function of peptide concentration did not change when the order of addition was varied, e.g., when topoisomerase was pre-incubated with peptides prior to the addition of the DNA substrate (data not shown). Kinetic analysis showed that the KWWWRW and KWWCRW peptides slowed the rate of transesterification.

To test whether the mechanism of topoisomerase inhibition necessitates direct interaction between the peptides and the DNA, we examined the effect of ionic strength on potency of the peptides. The potency of the KWWCRW peptide as an inhibitor of DNA cleavage by vaccinia topoisomerase was sensitive to changes in the ionic strength of the reaction mixture. Inclusion of 100 mM NaCl in the cleavage reactions resulted in a shift to the right in the peptide inhibition curve. Whereas 0.7 μM peptide reduced covalent adduct formation by 90% in the absence of added salt, the same concentration of peptide inhibited cleavage by only 40% in the presence of 100 mM NaCl. We noted a similar decrement in the potency of the KWWCRW and KWWWRW peptides in inhibiting relaxation for supercoiled plasmid DNA by vaccinia topoisomerase when the relaxation reaction mixtures were supplemented with 100 mM NaCl (data not shown). These results suggest that the peptide probably interacts with DNA as part of its inhibitory mechanism.

To test whether the peptide interferes with the noncovalent association of topoisomerase with the DNA, we assayed the effects of the peptides on the binding of vaccinia topoisomerase to a radiolabeled 60-bp duplex DNA containing a single central CCCTT recognition site. In contrast to the suicide substrate, for which all bound enzymes are trapped in the covalent state, only about 20% of the fully double-stranded DNA molecules that are bound will be linked covalently to the protein (Wittschieben and Shuman, 1997). Hence this gel shift assay largely reflects the noncovalent binding of enzyme to the DNA ligand. The most instructive finding was that concentrations of the KWWCRW peptide sufficient to block covalent adduct formation (0.72 to 1.8 μM peptide) did not inhibit formation of the noncovalent topoisomerase-DNA complex.

Peptide Inhibition of E. coli DNA Topoisomerase I

E. coli topoisomerase I (TopA) exemplifies the type IA topoisomerase family. Type IA enzymes are mechanistically and structurally unrelated to the topoisomerase IB/tyrosine recombinase superfamily of DNA strand transferases. Nonetheless, the relaxation of supercoiled DNA by E. coli TopA was inhibited in a concentration dependent manner by the KWWWRW and KWWCRW peptides. Activity was abolished at 15-42 μM peptide and reduced by one-half at approximately 7-10 μM peptide (Table 2). The other aromatic hexapeptides, WCHYNY and WKHYNY, had no effect on DNA relaxation by E. coli topoisomerase I at peptide concentrations up to 42 μM (data not shown). The specificity of peptide inhibition of DNA relaxation was similar for type IB and type IA topoisomerases, but the inhibitory peptides were about twice as potent on a molar basis against the type IB topoisomerase.

Inhibition of Type II Topoisomerase and Restriction Endonucleases

We further challenged the specificity of action of peptide KWWCRW by testing its effect on bacteriophage T4 topoisomerase, a type II enzyme. Indeed, KWWCRW inhibited T4 topoisomerase-induced DNA relaxation with an IC₅₀ of 40 μM and blocked it completely at 100 μM, while the similarly aromatic peptide WKHYNY had no effect on relaxation at 100 μM (Table 2). Because KWWCRW binds DNA, we also tested its effect on the activity of several restriction enzymes with unique sites in pUC19. Although each enzyme's recognition sequence contains a different distribution of A/T and G/C base pairs, all of the enzymes were inhibited with a similar IC₅₀, roughly 40 μM (Table 2). These results indicate that the peptide's DNA-binding property may interfere relatively nonspecifically with the activities of several DNA cutting enzymes. A summary of IC₅₀ values for the inhibition of DNA cleaving enzymes discussed here is given in Table 2.

Discussion

The detailed analysis of biochemical reactions depends on the ability to trap and study reaction intermediates. This has been particularly difficult in the case of reactions catalyzed by tyrosine recombinases, which are very efficient, freely reversible, and do not require any high energy cofactors. Cellular type IB topoisomerases are mechanistically similar to the tyrosine recombinases, and the analysis of their reactions with DNA has been aided by the availability of inhibitors such as camptothecin, which stabilizes a covalent reaction intermediate (Rothenberg, 1997). Such mechanistic inhibitors have not been available for the tyrosine recombinases or for the vaccinia virus topoisomerase. While netropsin, a minor groove binding compound, does block recombination by competing with Int and with IHF for interactions with their respective DNA binding sites, it has not been useful in trapping reaction intermediates.

In the current work we have characterized two peptide inhibitors of DNA cleavage by λ Integrase. These inhibitors were identified using a deconvolution process of combinatorial peptide libraries (see above Section E; Pinilla et al., 1998) and represent the first peptide inhibitors of tyrosine recombinases. The potency of the peptides differs for the different pathways of Int-mediated recombination (Table 2). In the case of the bent-L and straight-L pathways, the substrates are identical at the loci of strand cleavage (and elsewhere except for 3 base substitutions in the P′1 arm binding site), yet the peptides inhibit the bent-L pathway 3 fold more efficiently than the straight-L pathway. The peptides inhibit integrative recombination with a somewhat higher IC₅₀, 0.2 μM; the attP substrate has additional DNA binding sequences important for recombination and is supercoiled, while attB contains only the core sequences, which are almost identical among all 4 Int substrates. Excisive recombination substrates are very closely related to integrative recombination substrates, but the distribution of protein binding sites along the DNA is different (FIG. 9). Moreover, an additional accessory protein, Xis, is necessary for excision. This pathway is inhibited with an IC₅₀ of 1.1 μM. It appears unlikely that the minor differences in DNA sequence underlies the difference in IC₅₀ values in the 4 pathways. We conclude that the difference in potency of peptides KWWCRW and KWWWRW each pathway reflects differences among the pathways in the interactions of Int with the loci of strand cleavage. Int interactions could vary due to a combination of architectural, kinetic, and stability factors. Furthermore, the rate-limiting step may be distinct for each recombination pathway, and thus the mechanistic step targeted by the peptide may not have an equally large effect in all of the pathways. The basis of differences between the inhibitory potency of the peptides in each pathway are being investigated, and libraries are being screened for active peptides using the excision pathway.

We do not yet know the mechanism by which the peptides inhibit DNA cleavage, nor the exact nature of their target. Although the peptides clearly bind and probably deform double-stranded DNA into a conformation that prevents it from entering a polyacrylamide gel (data not shown), peptide inhibition of Int is resistant to as much as 1 μg of nonspecific competitor DNA (FIG. 12). This suggests that the target of the peptide is a specific complex of enzyme with its substrate, or requires that the DNA substrate be in some way deformed by Int. Although the peptide does slightly decrease Int binding to its arm sites, it does not prevent Int from making stable contacts with the core sites in the context of either early (UMC species) or synaptic (BMC species) recombination intermediates. Therefore, the peptide more specifically targets interactions between Int and DNA which are necessary for DNA cleavage. Indeed, cleavage of both excision substrates is inhibited by peptide KWWCRW (data not shown). One possibility is that Int, like Cre (Guo et al., 1999) locally kinks the DNA double helix at the site of cleavage prior to nucleophilic attack, resulting in the unstacking of 2 base pairs. This possibility is supported by 2 pieces of evidence: 1) the peptide has a somewhat higher affinity for single-stranded than for double-stranded DNA (data not shown); and 2) Int makes the bases at the loci of strand cleavage hypersensitive to dimethyl sulfate (Segall, 1998), which modifies single-stranded DNA more efficiently than double-stranded DNA. This model and the implication of an additional intermediate step in the mechanism of Int-mediated recombination is being tested in detail.

The KWWCRW and KWWWRW peptides also inhibit a related tyrosine recombinase, the Cre enzyme of bacteriophage P1, as well as the more distantly related but mechanistically similar vaccinia virus topoisomerase. Although the peptides were most effective at inhibiting the pathway with which we screened them, the IC₅₀ of the peptides for the vaccinia topoisomerase is in the same range as the IC₅₀ for Int in integration and excision (Table 2). Moreover, the peptide inhibits DNA cleavage even at concentrations which have no effect on the noncovalent complex between the vaccinia topoisomerase and its DNA substrate. Thus, as in the case of Int, the mechanism of cleavage inhibition appears specific to enzyme-substrate interactions necessary for catalysis. As might be expected for peptide inhibitors that bind to DNA, the KWWCRW and KWWWRW peptides are not entirely specific to enzymes that employ a type IB topoisomerase mechanism. For example, they inhibit, albeit with a lower potency, the action of E. coli topoisomerase I, an enzyme that cleaves DNA one strand at a time via a transient 5′-phosphotyrosine linkage and leaves a free 3′ OH group (Wang, 1996). This enzyme has been shown to bind preferentially to single-stranded DNA, and may cleave DNA via a single-stranded DNA intermediate. In addition, the two peptides inhibit the T4 topoisomerase, a type II enzyme that also uses a tyrosine in a nucleophilic attack on the DNA phosphodiester backbone, but with a much reduced potency (an IC₅₀ of 40 μM, which is as much as 2000 fold lower than the potency of Int inhibition; Table 2).

One possible explanation for the lower potency of the peptides for the T4 topoisomerase and the E. coli topoisomerase I is that these topoisomerases have multiple target sites in the plasmid substrates, at which they act with similar efficiency; the higher IC₅₀ may simply reflect the necessity for more peptides to interact with all of the available target sites. In contrast, DNA cleavage for Int, Cre, and vaccinia topoisomerase was assayed on substrates in which a single target site was available. Therefore, we tested the inhibitory effect of the peptides on cleavage by several restriction enzymes, each of which has a single target sequence in pUC19. Each of these enzymes was inhibited with a similar IC₅₀ (Table 2), despite the fact that their restriction sites have different A/T versus G/C content and different distribution of the A/T versus G/C base pairs. Thus, the peptides are significantly less potent against either the T4 topoisomerase or the restriction endonucleases, and may inhibit these enzymes as a consequence of relatively nonspecific interactions with DNA. We propose that the peptides inhibit DNA cleavage in two distinct ways: by interacting specifically with enzyme-DNA intermediates in the case of the tyrosine recombinases and the Vaccinia type Ib topoisomerase (and perhaps less efficiently in the case of the E. coli type Ia topoisomerase), and by interacting nonspecifically with DNA in the case of the T4 topoisomerase and restriction enzymes.

Our study has shown that specific hexameric peptides are potent inhibitors of DNA cleavage by tyrosine recombinases. The peptides are useful new tools for the analysis of the mechanism of site-specific recombination. In addition, these peptides inhibit DNA cleavage by the vaccinia type I topoisomerase. This result shows that site-specific recombination can be used effectively as a screen for inhibitors against enzymes with related biochemical mechanisms. Such approaches should continue to be useful as well-studied reactions by prokaryotic enzymes can be used to screen inhibitors of structurally and mechanistically related eukaryotic enzymes.

Materials and Methods

Proteins: Purified Int was the generous gift of C. Robertson and H. Nash (NIH), and of J. Hartley (Gibco BRL Life Technologies Inc.). Purified IHF was the generous gift of S.-W. Yang and H. Nash (NIH), while purified Xis was the generous gift of C. Robertson and H. Nash (NIH). HU was purified as described (Segall et al., 1996).

Vaccinia topoisomerase was expressed in Escherichia coli BL21 cells infected with bacteriophage λCE6 and then purified from a soluble bacterial lysate by phosphocellulose column chromatography (Shuman et al., 1988). The protein concentration of the phosphocellulose preparation was determined by using the dye-binding method (Biorad) with bovine serum albumin as the standard.

T4 topoisomerase was the generous gift of K. Kreuzer (Duke University). E. coli topoisomerase I was the generous gift of K. Marians (Memorial Sloan-Kettering Cancer Center). Cre protein and lox recombination substrates were generously provided by Alex Burgin. Restriction enzymes, VENT polymerase, and T4 polynucleotide kinase were purchased from New England BioLabs. γ-³²P-ATP was purchased from New England Nuclear.

DNA substrates for Int and T4 topoisomerase assays: Linear substrates for site-specific recombination or mobility shift assays were synthesized by PCR using plasmids with cloned attB, attL, attLtenP′1, attL-QH′, or attR sites and labeled at the 5′ end with [γ-³²P]ATP using T4 polynucleotide kinase as described (Segall et al., 1994). Supercoiled pUC19 for relaxation assays by T4 topoisomerase and pHN894 containing the attP substrate for integration were isolated from DH5α cells using the Qiagen Midi plasmid purification kit (Qiagen).

DNA substrates for vaccinia topoisomerase. DNA oligonucleotides were 5′ end-labeled by enzymatic phosphorylation in the presence of [γ-³²P]ATP and T4 polynucleotide kinase, then purified by preparative electrophoresis through a 15% polyacrylamide gel containing TBE (90 mM Tris-borate, 2.5 mM EDTA). The labeled oligonucleotides were eluted from an excised gel slice and then hybridized to unlabeled complementary oligonucleotide(s) as specified in the figure legends. Annealing reaction mixtures containing 0.2 M NaCl and oligonucleotides as specified were heated to 70° C. and then slow-cooled to 22° C. The hybridized DNAs were stored at 4° C.

Int Assays. Site-specific recombination and gel mobility shift assays were performed as described (Segall, 1998). Briefly, reactions were performed in a total volume of 10 or 20 μl and typically contained 1-2 nM radiolabeled att site as specified, 4 nM unlabeled att site, 100-300 ng salmon sperm DNA as nonspecific competitor, 44 mM Tris-Ci (pH 8.0), 60 mM KCl, 0.05 mg/ml bovine serum albumin, 7 mM Tris borate (pH 8.9), 5 mM spermidine, 1.3 mM EDTA, and 14.6% v/v glycerol. Any deviation from this formulation is noted in the figure legends. Gel shift reactions were incubated for 90 minutes at 37° C., layered without loading dyes onto 5% native polyacrylamide gel (29 acrylamide: 1 bis-acrylamide) and electrophoresed in 0.5× Tris borate EDTA buffer. Recombination reactions were stopped with 0.2× volume of 2% SDS, layered onto 5% polyacrylamide Tris/SDS gels, and electrophoresed in 1× Tris Tricine SDS buffer at 100 mA (Segall, 1998). Dried gels were visualized and quantitated using a Molecular Dynamics PhosphorImager.

Restriction enzyme assays. Restriction digests were performed as specified and the products were separated on 0.8% agarose gels electrophoresed at 80-90V in 1× Tris borate EDTA buffer. Gels were photographed, scanned and quantitated using NIH Image v.1.55, as recommended in the instruction manual.

T4 topoisomerase assays. Reactions were performed as described (Huff and Kreuzer, 1990) using 30 or 60 ng of enzyme and 200 ng of supercoiled pUC19 per reaction. The products were electrophoresed on 0.8% agarose gels at 40V in 0.5× Tris borate EDTA buffer for about 6 hours. The gel was then stained with EtBr for viewing.

Peptides. Peptides were synthesized with a C-terminal amide group using TBOC-protected amino acids (Pinilla et al., 1998), followed by HPLC-purification, at Torrey Pines Institute for Molecular Studies. The molar concentrations of the peptides KWWWRW, KWWCRW, WCHYNY, and WKHYNY were calculated from the absorbance at 280 nm at neutral pH using the extinction coefficients of 1.4×10³ M⁻¹ for tyrosine and 5.6×10³ M⁻¹ for tryptophan.

G. Analysis of Holliday junction Intermediates of Bacteriophage λ Site-Specific Recombination Using a Peptide Inhibitor

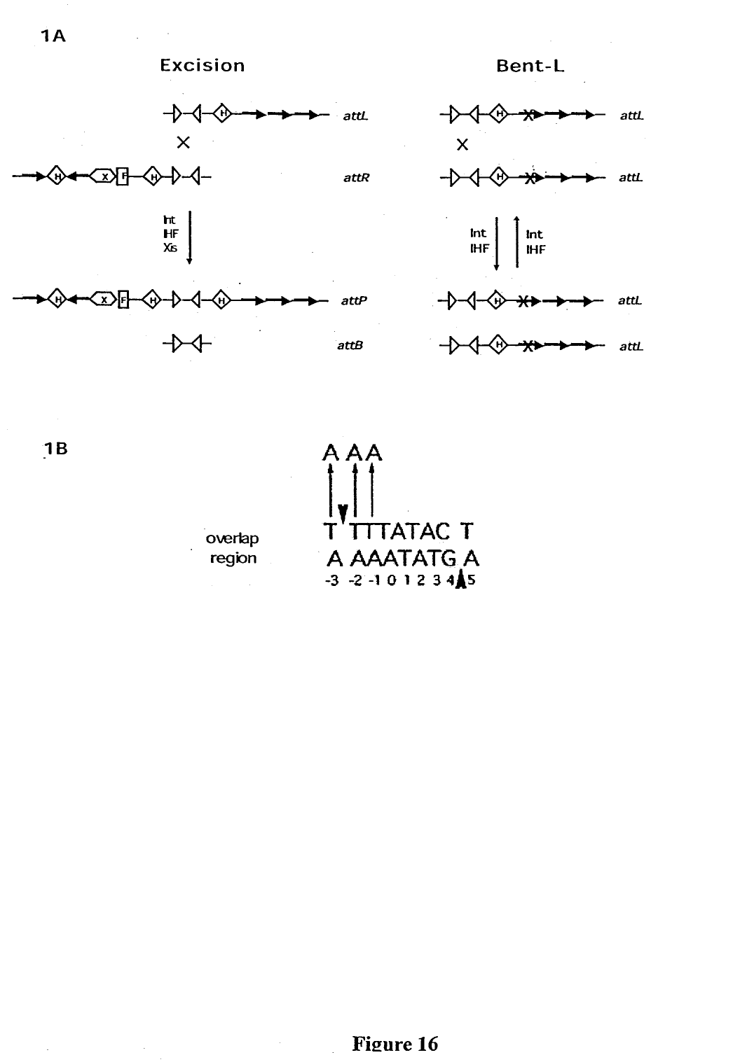

Site-specific recombination reactions are widespread in nature and are used to accomplish numerous biological functions, including control of gene expression, copy number amplification, creation of genetic diversity, and separation of chromosomes (reviewed by Nash, 1996; Landy, 1993). Many of these reactions, exemplified by recombination of bacteriophage P1 lox sites by the phage P1-encoded Cre recombinase, are random and bidirectional (the structure of the products is the same as that of the substrates) and the target sites of recombination are symmetrical. Some bacteriophages, exemplified by phage λ, use more complex recombination reactions to generate lysogens and later to resume lytic growth by excising the prophage from the host chromosome. These integrative and excisive recombination reactions are unidirectional, in which the structure of the products differs from that of the substrates (e.g., FIG. 16A). The phage λ site-specific recombinase, Integrase (Int), is aided by accessory factors encoded by the phage (Excisionase (Xis)) and by the host (Integration Host Factor (IHF) and Factor for Inversion Stimulation (FIS)). The ability of Int to act in the context of different pairs of recombination substrates is poorly understood at the molecular level.

Like its relatives Cre and Flp, Int also carries out bidirectional recombination reactions. One of these is the efficient bent-L pathway, which has been reconstituted in vitro (Segall and Nash, 1996; FIG. 16A). Higher-order intermediates in this pathway have been described and synapsis has been identified as the rate-limiting step in the reaction (Segall, 1998). The bent-L pathway has fewer requirements than integration or excision (it is Xis-, Fis- and supercoiling-independent; Segall and Nash, 1996; Table 3), although IHF is an absolute requirement for recombination. The pathway appears less stringent than integration or excision since several mutants of Int which are defective in these reactions remain proficient in the bent-L reaction (Segall and Nash, 1996). Therefore the bent-L pathway provides a unique context in which to separate the catalytic requirements of recombination from those features which control unidirectionality.

TABLE 3
The four pathways of phage λ Int-mediated site-specific recombination

| Pathway: | INTEGRATION | EXCISION | BENT-L | STRAIGHT-L |
|---|---|---|---|---|
| Substrates | attB, attP | attL, attR | attL¹ | attL |
| Requirements | Int, IHF, sc² attP | Int, IHF, Xis | Int, IHF | Int |
| Efficiency | high | high | high | low |
| Directionality | unidirectional | unidirectional | bidirectional | bidirectional |

¹Contains the attLtenP'1 mutations for in vitro analysis (Segall and Nash, 1996).
²attP must be supercolied, and is provided on a plasmid.

The catalytic steps of integration and excision have been characterized extensively (Kitts and Nash, 1987, 1988; Burgin and Nash, 1992, 1995; Nunes-Duby et al., 1987, 1995; Azaro and Landy, 1997; outlined in FIG. 16C). After integrase binds to its substrates, the top strands of each substrate are cleaved and then swapped to create a Holliday junction (HJ). Subsequent cleavage, exchange and ligation of the bottom strands resolve this HJ to recombination products. The identification of the rate-limiting step in the unidirectional pathways has been hampered by the fact that synaptic and Holliday junction intermediates in these pathways do not accumulate, due both to the high efficiency and the high reversibility of Int.

We have recently identified hexapeptide inhibitors of Int-mediated recombination, one of which, WKHYNY, causes the accumulation of Holliday junctions (see above Sections E and F). In the work presented here, we determined that peptide WKHYNY acts after the first round of Int-mediated DNA cleavage to stabilize protein-bound HJs. Using this peptide, we have characterized and compared HJ intermediates of the bent-L and excision pathways. Our analyses showed that strand exchange in bent-L recombination does not require the absolute order of strand exchanges observed in excisive recombination and that spermidine acts at the HJ resolution step in excision to bias the directionality of cleavage in favor of products rather than substrates.

Results

Holliday Junction Isolation and Characterization

The central intermediate of the tyrosine recombinase-mediated reactions is the Holliday junction. The processing of the integration and excision HJs has been studied using synthetic χ forms (Hsu and Landy, 1984; de Massy et al., 1989; Franz and Landy, 1990; 1995). However, these studies could not determine the kinetics of HJ appearance and disappearance as an intermediate of recombination. Moreover, the HJ in the bent-L pathway has not yet been examined.

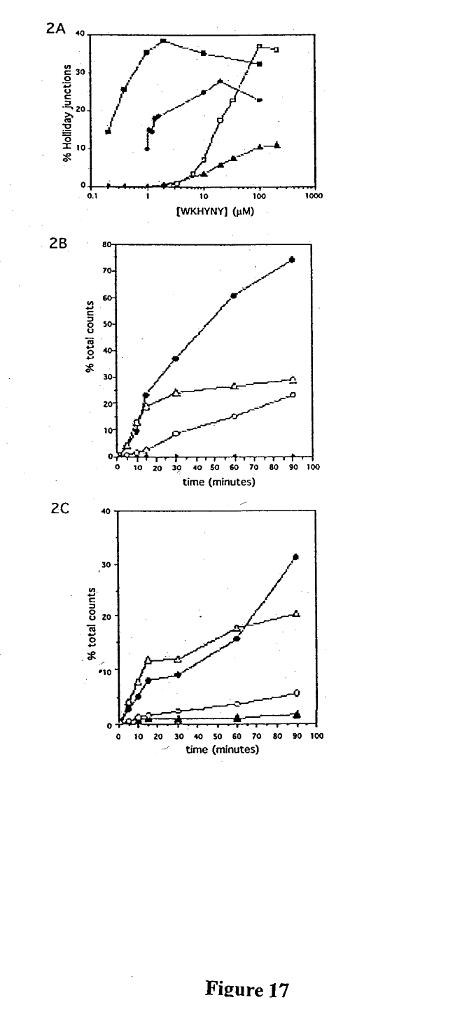

Since fewer than 1-2% HJs accumulate in a typical Int-mediated reaction, we used the hexapeptide WKHYNY to accumulate HJs for ease of analysis. The identification and initial characterization of this peptide is described in the above Section E. As expected for HJs, the species that accumulates on addition of peptide is resistant to proteinase K digestion and its mobility depends on the size of both substrates (see the above Section E). Addition of the peptide leads to accumulation of HJs in all λ site-specific recombination (SSR) pathways, albeit with different efficiencies; the half-maximal dose for HJ accumulation ranges from 0.2-0.4 μM for the bent-L pathway to 10-20 μM for excision (FIG. 17A). Timecourses were performed for these two pathways to follow the appearance of products with respect to the accumulation of the HJs both in the presence and absence of peptide WKHYNY (FIGS. 17B and 17C). In the absence of peptide, recombinant products increased over time to over 70% in excision and over 30% in bent-L recombination, whereas only a very low and constant level of HJs can be detected. In the presence of peptide, however, HJs appeared before recombinant products and accumulated over time, while the amount of substrate converted into recombinant products was reduced as compared to reactions not treated with peptide (FIGS. 17B and 17C). Thus WKHYNY acts relatively early during recombination, and appears to prevent the resolution of HJs, since they do not disappear at later time points. We show below that the peptide indeed slows the rate of HJ resolution.

Since the bent-L pathway differs significantly from the excisive pathway (FIG. 16A; Table 3), we isolated and characterized the excision and bent-L HJs in order to determine their respective strand composition. Strand composition indicates whether recombination was initiated at the top or at the bottom strand of the att substrates. In vitro excisive and bent-L recombination reactions containing a 5′ double end-labeled att site and a second unlabeled att site of different length were assembled and recombination products were separated on SDS-containing gels. In vitro, bent-L recombination is inhibited by Int bound at the P′1 arm site (Segall and Nash, 1993; Segall and Nash, 1996) and thus the bent-L substrates contain 3 base substitutions which prevent Int binding to P′1 (attLtenP′1; Numrych et al., 1990). The excision and bent-L HJs were eluted from the gel, concentrated, and electrophoresed on a DNA-denaturing gel. As expected for the excision HJs, only a substrate-length fragment and the product of top strand ligation were present (data not shown). In contrast, bent-L HJ intermediates contained the substrate-length fragment as well as fragments diagnostic of both top and bottom strand ligation (data not shown). However, top strand exchange was favored approximately 3:1. Thus, while excisive recombination initiated only at the top strand, it appears that bent-L recombination initiated either at the top or at the bottom strand.

We conclude that substrates in the bent-L pathway are processed in a more symmetric fashion than those in excision, since recombination starts with bottom rather than top strand exchange over 25% of the time. Moreover, peptide WKHYNY changes only the amount of HJ intermediates that accumulate and has no effect either on the order or the bias of strand exchanges, nor on the alignment of att substrates during synapsis.

saf Mutations Alter the Bias of Top Versus Bottom Strand Exchange

We wanted to test whether bottom strand exchange can occur in the absence of top strand exchange. To block top strand exchange, we paired a wild type substrate with a substrate that carries site affinity (saf) mutations at or near the top strand cleavage locus (saf-2A, saf-1A; FIG. 16B). Saf mutations, isolated by Weisberg and colleagues (Weisberg et al., 1983), are base substitutions in the overlap region of the att site that permit cleavage but prevent ligation to a wild type DNA partner (Burgin and Nash, 1995). In integrative and excisive reactions, the saf mutations near the locus of top strand cleavage blocked HJ formation as well as complete recombination (Kitts and Nash, 1987; Richet et al., 1988; Nash and Robertson, 1989). We predicted that these mutations should also block bent-L recombination, but not HJ formation if the latter can form either by bottom or top strand exchange. Indeed, both saf-2A and saf-1 A mutations reduced bent-L recombination with a wild type substrate approximately twenty-fold but decreased HJ formation only by approximately 33% (Table 4). Moreover, the saf mutations within the overlap region markedly altered the top strand exchange bias of bent-L recombination: the ratio was reversed in favor of bottom strand exchange products (Table 4). When the same T→A mutation was placed at position −3, just outside the overlap region, neither recombination nor HJ formation were affected, and no change in bias of strand exchanges was detected (Table 4). This agrees with data showing that homology sensing occurs within the overlap region, at the strand exchange stage of the reaction (Burgin and Nash, 1995; Nunes-Duby et al., 1995). Recombination performed between two substrates containing the same saf mutation gave wild type levels of recombinant products and HJs (data not shown), as expected for sites which would not generate heterology in the overlap region after strand exchange.

TABLE 4
Effect of saf mutations on strand exchange bias in
bent-L Holliday junctions

| Substrate | WT | -3A | saf-2A | saf-1A |
|---|---|---|---|---|
| % Recombination | 23 | 26 | 1 | 2 |
| % Holliday junction | 25 | 24 | 16 | 16 |
| top:bottom¹ | 3:1 | 4:1 | 1:2.5 | 1:3 |

¹Proportion of top strand exchange products to bottom strand exchange products within bent-L Holliday junctions

In conclusion, while heterologies within the overlap region profoundly decrease recombination in the excision, integration and bent-L pathways (Table 4 and Kitts and Nash, 1988; R. Weisberg, pers. commun.), they only moderately reduce HJs in the bent-L pathway. This confirms that bent-L HJs can form via bottom strand exchange in the absence of completed top strand exchange.

Holliday Junction Resolution

Holliday junctions can be resolved either in the “forward” direction to form recombinant products or in the “reverse” direction to re-form substrates. Synthetic HJs representing intermediates in the unidirectional reactions are preferentially resolved in the direction of products (Hsu and Landy, 1984; Franz and Landy, 1990, 1995). We wanted to determine whether HJs isolated in the presence of peptide behave similarly to the synthetic HJs. In addition, we wanted to analyze the processing of bent-L HJs. Recombination reactions were assembled in the presence of peptide WKHYNY, and protein-free HJs were isolated from SDS-containing gels, eluted, and precipitated. Resolution reactions were then performed following the same protocol as for in vitro recombination reactions, but replacing the att site DNA substrates with the HJs.

We first tested the binding of recombination proteins to HJs. IHF and Int bound individually to both excision and bent-L HJs (data not shown). Although Xis did not change the mobility of the excision HJ by itself, it did contribute to the formation of specific complexes whose mobility depends on all three proteins (data not shown). Moreover, the 3 proteins indeed efficiently resolved the excision HJs (Table 5). As documented for synthetic HJs (Franz and Landy, 1990), Int was sufficient for resolution of excision HJs; in part, Int alone may resolve only a small fraction of HJs because it does not bind well by itself to HJs of either pathway (data not shown). Addition of Xis alone, and particularly IHF alone, stimulated resolution (by 2- and 5-fold respectively; Table 5). Xis and IHF together additively stimulate resolution of HJs by Int (Table 5). Moreover, the presence of both accessory proteins affected the resolution bias of the HJs. Int alone generated recombinant products and substrates in roughly equal proportions. While the addition of Xis did not affect the direction of resolution, addition of IHF favored products over substrates 2:1. Maximum bias towards products was achieved only when both IHF and Xis were present in addition to Int (Table 5). These results agree with the results obtained by Franz and Landy (1995) using artificially assembled HJs. Thus, we conclude that HJs isolated using peptide WKHYNY retain the basic properties expected for intermediates of Int-mediated recombination.

TABLE 5
Resolution of excision HJs in the absence of peptides.

| Conditions | % Resolution | products/substrates |
|---|---|---|
| Int | 7.2 ± 0.4 | 1.3 |
| Int + IHF | 42.4 ± 3.8 | 2.5 |
| Int + Xis | 14.4 | 1.2 |
| Int + IHF + Xis | 58.2 ± 9.9 | 5.1 |
| Int + IHF + Xis (no spermidine) | 95.3 | 1.4 |

A long-standing observation for λ Int-mediated recombination is that spermidine stimulates the reaction about 5 fold (Nash, 1975), but is not necessary for assembly of early intermediates nor synaptic complexes between DNA substrates (Segall and Nash, 1993; Segall et al., 1994; Segall, 1998). It is still unknown at what stage spermidine exerts its effect. Interestingly, spermidine inhibited resolution of synthetic HJs somewhat (Hsu and Landy, 1984) and our data confirmed this (Table 5). However, we have found that spermidine strongly affected the bias of HJ resolution: in the absence of spermidine, resolution was essentially equal towards products or substrates, while in the presence of spermidine resolution favored products about 5 fold (Table 5). Thus overall recombination efficiency may be sacrificed somewhat in order to ensure that the reaction proceeds to completion.

We also tested the resolution of bent-L HJs. However, in contrast to the excision HJs, bent-L HJs which were isolated and re-loaded with proteins were not resolved (either in the presence or absence of spermidine; data not shown), despite the fact that both Int and IHF bound to the HJs (data not shown). These HJ complexes were quite stable and were not destroyed by branch migration (data not shown). We ruled out irreversible modification of the HJs during the isolation protocol by showing that the bent-L HJs were sensitive to digestion with Hinf I and Bsr DI, restriction enzymes that have recognition sequences adjacent to the core and within the H′ IHF binding site of the attL substrate, respectively (data not shown). Resistance to Int-mediated resolution suggests that the conformation of the HJ generated during the recombination process cannot be replicated by de novo binding to the deproteinated HJs—some feature established at or shortly after the beginning of the reaction cannot be established on newly loaded HJ substrates. We are analyzing the basis of this conformational feature further.

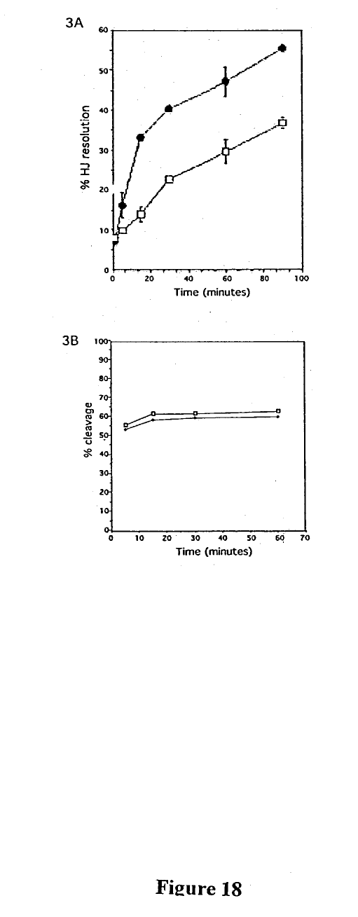

In analyzing the resolution of gel-isolated excision HJs, we also tested the effect of peptide WKHYNY on processing of the HJs. A comparison of the rate of resolution in the presence versus the absence of peptide showed that the peptide slowed the rate of cleavage (FIG. 18A). This effect presumably accounts at least in part for the HJ accumulating-activity of the peptide during recombination.

In contrast, the peptide has little or no effect on the, rate of cleavage of the attL early intermediate under recombination conditions (FIG. 18B). We measured this by using attL substrates containing a bridging phosphorothiolate modification at the locus of top strand cleavage. This modification, developed by Burgin and Nash (1995), replaces a bridging oxygen atom in the DNA backbone with a sulfur atom. Upon cleavage, the covalent Int-DNA intermediate remains trapped because the sulfhydryl generated at the free 5′ end is a much poorer nucleophile of the phosphotyrosyl bond than the normal hydroxyl group (Burgin et al., 1995). HJ formation is inhibited because at least one of strands (the one containing the sulfur) cannot be ligated. Int, IHF, and Xis were incubated with the attLS substrate in the presence of attR with or without peptide WKHYNY, and DNA cleavage was followed over time. The first cleavage event was very fast: over 50% of the site was cleaved within the first 5 minutes, and the peptide had no influence on the kinetics or the amount of cleavage. The same analysis was done for the bent-L pathway on attLtenP′1 sites with similar results (data not shown). The results agree with our data that the peptide exerts its effect after strand cleavage.

Peptide WKHYNY Stabilizes Protein-Bound HJs

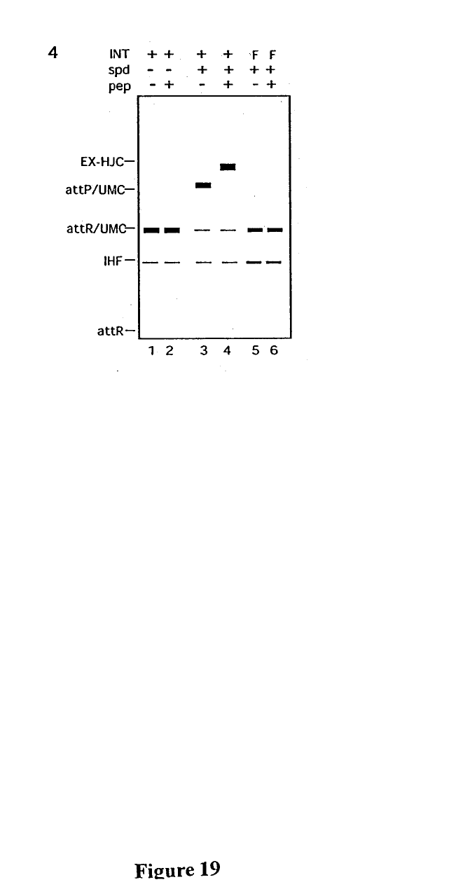

During our initial screen for the peptide and the earlier experiments described here, HJs were detected on SDS-containing gels. We investigated whether peptide WKHYNY causes the accumulation of “naked” HJs by somehow dissociating proteins from the HJ, or if it interacts with and stabilizes the protein-bound HJ. Excisive and bent-L recombination reactions were assembled with the appropriate att substrates, Int, and accessory proteins and incubated in the presence or absence of peptide. Protein-DNA complexes were then separated in the first dimension on a native polyacrylamide gel. A lane containing intermediates of each reaction was excised from the gel, layered on top of a protein-denaturing gel and electrophoresed in the second dimension to determine the DNA composition of the nucleoprotein complexes. In the absence of peptide, the resulting excision product on the native gel is attP, presumably bound by Int, IHF, and Xis (FIG. 19, lane 3), as verified by electrophoresis in the second dimension (data not shown). The attB migrated off this gel, but has been seen on other gels (data not shown))

In the presence of peptide, a new, slower excision complex accumulates on the native gel (FIG. 19, lane 4). This complex is dependent on strand cleavage: it does not form when Int is replaced with IntF, the catalytically defective IntY342F mutant protein (FIG. 19, lanes 5-6). It also does not form in the absence of spermidine (FIG. 19, lanes 1-2); the role of spermidine will be discussed in detail below. Two-dimensional analysis of this new complex showed that it contains radiolabeled attR substrate and some HJs, but mostly recombinant attP and attB products (data not shown). Based on the phenotype of the peptide during deconvolution (see above Section E), we were surprised that we did not trap a majority of HJs rather than recombination products. We reasoned that peptide WKHYNY may have dissociated from the complex either because of dilution during loading or during electrophoresis of the native gel, allowing Int, IHF and Xis to resolve the HJs to products. We confirmed this possibility by assembling recombination reactions and first trapping HJs with peptide WKHYNY, and after 30 minutes adding a second peptide, KWWCRW, which inhibits DNA cleavage by Int and some topoisomerases (see above Section F). The latter peptide interacts quite stably with Int-DNA complexes both in solution and during electrophoresis presumably because, unlike peptide WKHYNY, peptide KWWCRW binds double stranded DNA by itself (see above Section F). Indeed, addition of the second peptide resulted in a larger fraction of HJs and fewer recombinant products within the slow protein-DNA complex (data not shown).

Thus, we conclude that peptide WKHYNY stabilizes protein-bound HJs rather than disrupting the protein-bound HJs to generate the protein-free form. Based on this analysis, we have named this newly identified intermediate of excision the EX-HJC (excision HJ complex). This complex represents the first instance in which a stable nucleoprotein intermediate of excision containing both DNA recombination partners has been visualized.

In the case of the bent-L pathway, synaptic complexes containing the two DNA partners noncovalently joined have been identified and named the BL-BMC (Segall, 1998). While BL-BMCs form in the absence of peptide, the presence of peptide causes a greater accumulation of the complex in these reaction conditions (data not shown). In agreement with previous data (Segall, 1998), two-dimensional analysis shows that the BL-BMC contains both substrate and recombinant products in the absence of peptide (data not shown). In addition to these constituents, the BL-BMCs isolated from peptide WKHYNY-treated reactions also contained HJs (data not shown), and the proportion of HJs increased when cleavage was blocked subsequently with the more stably-interacting peptide KWWCRW (data not shown).

Discussion

The Holliday junction is the central intermediate for the reciprocal, conservative site-specific recombination reactions mediated by the tyrosine recombinase subclass of enzymes. Previous studies of these intermediates have used synthetic Holliday junctions assembled in vitro from constituent DNA strands (Hsu and Landy, 1984; de Massy et al., 1992; Franz and Landy, 1990, 1995) because this stage of the reaction is transient and few if any of these intermediates can be seen either in reactions mediated by wild type or by mutant Int proteins (Kitts and Nash, 1987, 1988). We have recently identified peptide inhibitors which cause the accumulation of Holliday junctions in Int-mediated recombination (see above Section E). Previous experiments have shown that these peptides, exemplified by peptide WKHYNY, do not bind double-stranded DNA, and do not inhibit cleavage by the mechanistically- and structurally-related type Ib topoisomerase encoded by Vaccinia virus (see above Section F). The peptide does cause accumulation of Holliday junctions in Cre-mediated recombination as well. Here we show that the peptide does not appreciably affect Int-mediated DNA cleavage, but exerts its effect after the first strand cleavage event and inhibits the resolution of pre-formed Holliday junctions. While it is formally possible that the peptide selectively inhibits the second strand exchange event rather than the first, we disfavor this interpretation for three reasons. First, in the bent-L pathway, we showed that Holliday junctions form either via top or bottom strand exchange; if the peptide selectively inhibited the second strand cleavage event, we would only see HJs formed via top strand exchange. Second, although the presence of the peptide slows HJ resolution, it does not affect the bias of strand cleavage events (data not shown). Finally, inhibition of the second strand cleavage event by using DNA modifications of several types have resulted in reversal of catalytic events to starting substrates rather than in the trapping of Holliday junctions (Kitts and Nash, 1987, 1988; Burgin and Nash, 1992; Nunes-Duby et al., 1995), which is why the peptide is proving so useful.

The peptide stabilizes protein-bound Holliday junction complexes, either when added at the beginning of recombination or when added to preformed junctions. Therefore, we think that they bind to the Holliday junction intermediate after it forms. We do not know whether binding and trapping of HJs absolutely requires that both strands have been ligated, although we know that ligation of one strand is sufficient for trapping HJs since we find proteinase-K sensitive complexes that migrate above the HJ position. The most likely target for the peptides is the center of the Holliday junction structure; crystal structures of both the Cre and the Flp proteins bound to Holliday junctions show a central opening with largely single-stranded character (Gopaul et al., 1998). Indeed, KMnO₄ footprints of the Int-bound Holliday junction intermediates show a hypersensitive signal in the core of the att sites which is not present in the double-stranded att substrates.

Peptide WKHYNY stabilizes Holliday junctions in all four pathways of Int-mediated recombination, but it does so with different potency. It is most effective in the bent-L pathway, the pathway originally used in the screen to identify the peptide, and least effective in the straight-L pathway, with intermediate potency in integration and excision. We believe that this reflects differences in the conformation of the Holliday junction intermediate in each of these pathways, and we are currently investigating these differences. The straight-L pathway requires a single protein, Int itself, but is the least inhibited. The low overall level of Holliday junctions that accumulate in this pathway is certainly a reflection of the low substrate turnover in this pathway. Thus it is unlikely that the peptide specifically interacts with one of the accessory proteins, but it is highly possible that the accessory proteins

In these studies of the Holliday junctions, we were able to compare the strand composition of junctions of the bent-L pathway with those of the excision pathway. Surprisingly, this analysis showed that the bent-L pathway can be initiated either by top strand exchange or, in a significant proportion of reactions, by bottom strand exchange. In contrast, both integrative and excisive recombination initiate exclusively by top strand exchange (FIG. 18; Kitts and Nash, 1987; 1988). This again highlights the differences in the conformation of nucleoprotein intermediates in each pathway, and suggests to us that it is the unique conformation that triggers the activation of the catalytic domain of Int monomers rather than an obligatory Int-DNA interaction in all pathways. We find it remarkable that a single enzyme displays the range of flexibility shown by the phage λ integrase.

Holliday junction substrates isolated from peptide-treated excision reactions and re-loaded with proteins behave similarly to artificially-assembled Holliday junctions. They are resolved either to products or to substrates. Int alone can accomplish resolution but is stimulated by Xis and particularly by IHF. However, directionality—the preferential resolution of junctions towards products—is most pronounced only in the presence of both accessory proteins and, we have found, only in the presence of spermidine. It is particularly striking that, in the absence of spermidine, resolution is almost twice as efficient but almost entirely bidirectional (Table 5). Interestingly, protein-free bent-L Holliday junctions can be re-loaded with proteins but are not resolved by Int in the presence or absence of IHF, nor with or without spermidine. We interpret this observation to indicate that the intermediates reassembled in vitro lack a conformational feature which must be established earlier during the recombination reaction, and which is necessary for Holliday junction resolution. This feature may be akin to the “molecular spring” feature invoked by Kleckner and colleagues to explain the progression of the Tn10 transpososome through some of its conformational stages (Chalmers et al., 1998). Comparing the fine structure of EX-HJC intermediates isolated on native gels with the structure of in vitro loaded HJs will provide insight into this issue. However, we are alerted that the structure and processing of artificial HJs may not filly reflect the structure and processing of the actual Holliday junction intermediates generated during recombination.

Materials and Methods

DNA Substrates

att sites: The att site recombination substrates were generated by PCR with Vent polymerase (New England Biolabs) using the following plasmids as templates: pHN872 for attL; pHN868 for attR, and pHN1679 for attLtenP′1. Fifty pmol oligos were labeled with 50 μCi γ³²P-ATP (New England Nuclear) and 15 units T4 polynucleotide kinase (NEB) at 37° C. for 60 min. The unincorporated nucleotides were removed through a P6 spin column (Bio Rad). PCR was carried out with thirty cycles of melting at 95° C. for 30 sec, annealing at 60° C. for 1 min, and extension at 72° C. for 1 min. PCR products were separated via 5% PAGE in 0.5×TBE at 100 V for 5 hours. The appropriate band was excised from the gel and eluted overnight in TE at 37° C. The DNA was then ethanol precipitated in the presence of 1/10 volume potassium acetate (Sigma).

Proteins: Int protein was purified as described by Nash and Robertson, with the following modifications: IHF protein was the generous gift of Shu-Wei Yang and Howard Nash. Xis was expressed in BL21 (λ DE3) cells from a clone graciously provided by Steven Goodman and purified as a 6× His-tagged protein using immobilized metal affinity chromatography with cobalt-loaded resin (Clonetech).

Modified att sites: Oligonucleotides containing the T→A sequence changes and the phosphorothiolate modification in the overlap regions of attL sites were synthesized at the SDSU Microchemical Core Facility. These were then used as primers in PCR reactions to generate the attL or attL tenP′1 DNA substrates (see above). We are extremely grateful for the phosphorothiolate-modified phosphoramidite synthesized by Alex Burgin, Jr.

In Vitro Recombination

Excision reactions: Reactions were performed in 10 μL volume containing 20 mM Tris-HCl pH 8, 5 mM spermidine, 0.2 μg BSA, 75 ng to 0.15 μg salmon sperm DNA, 30 mM KCl, and TE (recombination mix). DNA and proteins were added to final concentrations of: 55 nM Int, 35 nM IHF, 50 nM Xis, 1 nM radiolabeled attL, and 4 nM unlabeled attR. Peptide WKHYNY-NH₂, a generous gift from Clemencia Pinilla, was synthesized at Torrey Pines Institute for Molecular Studies and was added to 100 μM. The reactions were incubated at room temperature for 60 to 90 min. The reactions were stopped with the addition of loading dye (2% SDS/xylene cyanol) and separated on a 5% polyacrylamide/0.1%. SDS gel in Tris/Tricine/SDS buffer at 100 mA for 3 to 5 hours (29:1 ratio of acrylamide:bis-acrylamide; DocFrugals). All gel images were visualized with a PhosphorImager (Molecular Dynamics) and quantitated with ImageQuant software (Molecular Dynamics).

Bent-L reactions: Reactions were performed as for excision, with the following exceptions: reactions contained 1 nM radiolabeled attLtenP′1, 4 nM unlabeled attLtenP′1, 10 μM peptide 52, and were incubated at 30° C.

Bandshift Reactions

Bandshift reactions were performed exactly as the in vitro recombination but were directly loaded onto a 5% native polyacrylamide gel without any stop buffer or loading dye. Electrophoresis was performed at 240V in 0.5×TBE at 4° C. for approximately 3 hours.

Two-Dimensional Gel Electrophoresis

Holliday intermediates: Gel slices corresponding to Holliday intermediates were isolated from an SDS protein-denaturing gel. The DNA was eluted in 500 μL TE at 37° C. overnight, then ethanol-precipitated with 1/10 volume potassium acetate and 2 μg tRNA at −80° C. for about 6 hours. Pellets were resuspended in 10 μL recombination mix. Proteinase digestions were carried out in the presence of 0.25% SDS and 0.25 μg proteinase K (Sigma) at 37° C. for 1 hour. One volume of sequencing loading dye (15% Ficoll/xylene cyanol/bromphenol blue) was added, and the samples were boiled for 5 min prior to electrophoresis. DNA-denaturing gels containing 7 M urea and 6% polyacrylamide were pre-electrophoresed for 20 min, loaded and electrophoresed for 60 to 90 min at 600 V with 0.5×TBE in the upper buffer chamber and 1×TBE in the lower chamber.

Synaptic intermediates: Gel slices containing bimolecular complexes were isolated from native gels, soaked in 2% SDS/xylene cyanol, and loaded onto an SDS protein-denaturing gel and electrophoresed at 100 mA in Tris/Tricine/SDS buffer for 5 hours.

### References

Altschul, S. F., T. L. Madden, A. A. Schäffer, J. Zhang, Z. Zhang, W. Miller, & D. J. Lipman (1997), Gapped BLAST and PSI-BLAST: a new generation of protein database search programs, Nucl. Acids Res. 25, 3389-3402.

Appel, J. R., Buencamino, J., Houghten, R. A. & C. Pinilla (1996). Exploring antibody polyspecificity using synthetic combinatorial libraries. Molec. Div., 2, 29-34.

Azaro, M. A. & Landy, A. (1997). The isomeric preference of Holliday junctions influences resolution bias by lambda integrase. EMBO J 16(12), 3744-55.

Burgin, A. B., Jr., Huizenga, B. N. & Nash, H. A. (1995). A novel suicide substrate for DNA topoisomerases and site-specific recombinases. Nucleic Acids Res 23(15), 2973-9.

Burgin, A. B., Jr. & Nash, H. A. (1992). Symmetry in the mechanism of bacteriophage lambda integrative recombination. Proc Natl Acad Sci USA 89(20), 9642-6.

Burgin, A. B., Jr. & Nash, H. A. (1995). Suicide substrates reveal properties of the homology-dependent steps during integrative recombination of bacteriophage lambda. Curr Biol 5(11), 1312-21.

Bushman, W., Thompson, J. F., Vargas, L. & Landy, A. (1985). Control of directionality in lambda site-specific recombination. Science 230, 906-911.

Cassell, G., Klemm, M., Pinilla, C. & Segall, A. (2000). Dissection of bacteriophage_site-specific recombination with synthetic peptide combinatorial libraries. J. Mol. Biol. 299, 1193-1202.

Cassell, G., Moision, R., Rabani, E. & Segall, A. (1999). The geometry of a synaptic intermediate in a pathway of bacteriophage lambda site-specific recombination. Nucleic Acids Res 27, 1145-51.

Cheng, C., Kussie, P., Pavletich, N. & Shuman, S. (1998). Conservation of structure and mechanism between eukaryotic topoisomerase I and site-specific recombinases. Cell 92, 841-850.

de Massy, B., Dorgai, L. & Weisberg, R. A. (1989). Mutations of the phage lambda attachment site alter the directionality of resolution of Holliday structures. EMBO J., 8, 1591-9.

Dooley, C. T., Spaeth, C. G., Berzetei-Gurske, I. P., Craymer, K., Adapa, I. D., Brandt, S. R., Houghten, R. A. & Toll, L. (1997). Binding and in vitro activities of peptides with high affinity for the nociceptin/orphanin FQ receptor, ORL1. J Pharm. Exp. Therap., 283, 735-741.

Esposito, D. & Scocca, J. J. (1997). The integrase family of tyrosine recombinases: evolution of a conserved active site domain. Nucl. Acids Res, 25, 3605-3614.

Franz, B. & Landy, A. (1990). Interactions between lambda Int molecules bound to sites in the region of strand exchange are required for efficient Holliday junction resolution. J Mol Biol 215, 523-35.

Franz, B. & Landy, A. (1995). The Holliday junction intermediates of lambda integrative and excisive recombination respond differently to the bending proteins integration host factor and excisionase. EMBO J 14, 397-406.

Gardner, J. F. & Nash, H. A. (1986) Role of Escherichia coli IHF protein in lambda site-specific recombination. A mutational analysis of binding sites. J. Mol. Biol., 191, 181-189.

Gopaul, D. N. & Duyne, G. D. (1999). Structure and mechanism in site-specific recombination. Curr Opin Struct Biol 9, 14-20.

Gopaul, D. N., Guo, F. & Van Duyne, G. D. (1998). Structure of the Holliday junction intermediate in Cre-loxP site-specific recombination. EMBO J 17, 4175-87.

Guo, F., Gopaul, D. N. & van Duyne, G. D. (1997). Structure of Cre recombinase complexed with DNA in a site-specific recombination synapse. Nature 389, 40-6.

Guo, F., Gopaul, D. N. & Van Duyne, G. D. (1999). Asymmetric DNA bending in the Cre-loxP site-specific recombination synapse. Proc Natl Acad Sci USA 96, 7143-8.

Han, Y. W., Gumport, R. I. & Gardner, J. F. (1993). Complementation of bacteriophage lambda integrase mutants: evidence for an intersubunit active site. EMBO J, 12, 4577-4584.

Han, Y. W., Gumport, R. I. & Gardner, J. F. (1994). Mapping the functional domains of bacteriophage lambda Integrase protein. J. Mol. Biol., 235, 908-925.

Houghten, R. A., Pinilla, C., Appel, J. R., Blondelle, S. E., Dooley, C. T., Eichler, J., Nefzi, A. & Ostresh, J. M. (1999) Mixture-based synthetic combinatorial libraries. J. Med. Chem., 42, 3743-3778.

Houghten, R. A., Pinilla, C., Blondelle, S. E., Appel, J. R., Dooley, C. T. & Cuervo, J. H. (1991) Generation and use of synthetic peptide combinatorial libraries for basic research and drug discovery. Nature, 354, 84-86.

Hsu, P. L. & Landy, A. (1984). Resolution of synthetic att-site Holliday structures by the integrase protein of bacteriophage lambda. Nature 311, 721-6.

Huff, A. C. & Kreuzer, K. N. (1990). Evidence for a common mechanism of action for antitumor and antibacterial agents that inhibit type II DNA topoisomerases. J. Biol. Chem. 265, 20496-20505.

Kho, S. H. & Landy, A. (1994). Dissecting the resolution reaction of lambda integrase using suicide Holliday junction substrates. EMBO J 13, 2714-24.

Kikuchi, Y. and H. Nash. 1979. Nicking-closing activity associated with bacteriophage I int gene product. Proc. Natl. Acad. Sci. USA 76, 3760-3764.

Kitts, P. A. & Nash, H. A. (1987). Homology-dependent interactions in phage lambda site-specific recombination. Nature 329, 346-8.

Kitts, P. A. & Nash, H. A. (1988a). Bacteriophage lambda site-specific recombination proceeds with a defined order of strand exchanges. J Mol Biol 204, 95-107.

Kitts, P. A. & Nash, H. A. (1988b). An intermediate in the phage lambda site-specific recombination reaction is revealed by phosphorothioate substitution in DNA. Nucleic Acids Res 16, 6839-56.

Klemm, M., Cheng, C., Cassell, G., Shuman, S. & Segall, A. (2000). Peptide inhibitors of DNA cleavage by tyrosine recombinases and topoisomerases. J. Mol. Biol., 299, 1203-.

Landy, A. (1989). Dynamic, structural, and regulatory aspects of lambda site-specific recombination. Annu. Rev. Biochem. 58, 913-949.

Landy, A. (1993). Mechanistic and structural complexity in the site-specific recombination pathways of Int and FLP. Curr Opin Genet Dev 3, 699-707.

Lowman, H. B. (1997). Bacteriophage display and discovery of peptide leads for drug development. Annu. Rev. Biophys. Biomol. Struct., 26, 401-424.

Nash, H. A. (1975). Integrative recombination of bacteriophage lambda DNA in vitro. Proc Natl Acad Sci USA, 72, 1072-6

Nash, H. A. (1996). Site-specific recombination: Integration, excision, resolution, and inversion of defined DNA segments, pp. 2363-2376. In F. C. Neidhardt (ed. in chief) Escherichia coli and Salmonella: Cellular and Molecular Biology. ASM Press, Washington D.C.

Nash, H. A., Bauer, C. E. & Gardner, J. F. (1987). Role of homology in site-specific recombination of bacteriophage lambda: evidence against joining of cohesive ends. Proc Natl Acad Sci USA 84, 4049-53.

Nash, H. A. & Robertson, C. A. (1989). Heteroduplex substrates for bacteriophage lambda site-specific recombination: cleavage and strand transfer products. EMBO J 8, 3523-33.

Numrych, T. E., Gumport, R. I. & Gardner, J. F. (1990). A comparison of the effects of single-base and triple-base changes in the integrase arm-type binding sites on the site-specific recombination of bacteriophage lambda. Nucl. Acids Res., 18, 3953-3959.

Nunes-Duby, S. E., Azaro, M. A. & Landy, A. (1995a). Swapping DNA strands and sensing homology without branch migration in lambda site-specific recombination. Curr Biol 5, 139-48.

Nunes-Duby, S. E., Kwon, H. J., Tirumalai, R. S., Ellenberger, T. & Landy, A. (1998). Similarities and differences among 105 members of the Int family of site-specific recombinases. Nucl. Acids Res., 26, 91-106.

Nunes-Duby, S. E., Matsumoto, L. & Landy, A. (1987). Site-specific recombination intermediates trapped with suicide substrates. Cell 50, 779-88.

Nunes-Duby, S. E., Tirumalai, R. S., Dorgai, L., Yagil, E., Weisberg, R. A. & Landy, A. (1994). Lambda integrase cleaves DNA in cis. EMBO J., 13, 4421-4430.

Pargellis, C. A., Nunes-Duby, S. E., Moitoso de Vargas, L. & Landy, A. (1988). Suicide recombination substrates yield covalent lambda integrase-DNA complexes and lead to identification of the active site tyrosine. J Biol Chem 263, 7678-85.

Pinilla, C., Appel, J. R., Blanc, P. & Houghten, R. A. (1992) Rapid identification of high affinity peptide ligands using positional scanning synthetic peptide combinatorial libraries. Biotechniques, 13, 901-905.

Puras Lutzke, R. A., Eppens, N. A., Weber, P. A., Houghten, R. A. & Plasterk, R. H. L. (1995). Identification of a hexapeptide inhibitor of the human immunodeficiency virus integrase protein by using a combinatorial chemical library. Proc. Natl. Acad. Sci. USA, 92, 11456-11460.

Redinbo, M. R., Champoux, J. J. & Hol, W. G. J. (1999). Structural insights into the function of type IB topoisomerases. Curr. Opin. in Struct. Biol., 9, 29-36.

Redinbo, M. R., Stewart, L., Kuhn, P., Champoux, J. J. &Hol, W. G. (1998). Crystal structures of human topoisomerase I in covalent and noncovalent complexes with DNA. Science, 279, 1504-1513.

Richet, E., Abcarian, P. & Nash, H. A. (1988). Synapsis of attachment sites during lambda integrative recombination involves capture of a naked DNA by a protein-DNA complex. Cell 52, 9-17.

Rothenberg, M. L. (1997) Topoisomerase I inhibitors: review and update. Ann. Oncology, 8, 837-855.

Segall, A. M. (1998). Analysis of higher order intermediates and synapsis in the bent-L pathway of bacteriophage lambda site-specific recombination. J Biol Chem 273, 24258-65.

Segall, A. M., Goodman, S. D. & Nash, H. A. (1994). Architectural elements in nucleoprotein complexes: interchangeability of specific and non-specific DNA binding proteins. EMBO J 13, 4536-48.

Segall, A. M. & Nash, H. A. (1993). Synaptic intermediates in bacteriophage lambda site-specific recombination: integrase can align pairs of attachment sites. EMBO J 12, 4567-76.

Segall, A. M. & Nash, H. A. (1996). Architectural flexibility in lambda site-specific recombination: three alternate conformations channel the attL site into three distinct pathways. Genes Cells 1, 453-63.

Sekiguchi, J., Cheng, C., & Shuman, S. (1997) Kinetic analysis of DNA and RNA strand transfer reactions catalyzed by vaccinia topoisomerase. J. Biol. Chem. 272, 15721-15728.

Shuman, S., Golder, M., & Moss, B. (1988) Characterization of vaccinia virus DNA topoisomerase I expressed in Escherichia coli. J. Biol. Chem. 263, 16401-16407.

Shuman, S., & Prescott, J. (1990) Specific DNA cleavage and binding by vaccinia virus DNA topoisomerase I. J. Biol. Chem. 265, 17826-17836.

Stewart, L., Redinbo, M. R., Qiu, X., Hol, W. G. & Champoux, J. J. (1998). A model for the mechanism of human topoisomerase I. Science, 279, 1534-1541.

Tuerk, C. & Gold, L. (1990). Systematic evolution of ligands by exponential enrichment: RNA ligands to bacteriophage T4. Science, 249, 505-510.

Wang, J.C. (1996) DNA topoisomerases. Annu. Rev. Biochem., 65, 635-692.

Weisberg, R. A., Enquist, L. W., Foeller, C. & Landy, A. (1983). Role for DNA homology in site-specific recombination. The isolation and characterization of a site affinity mutant of coliphage lambda. J Mol Biol 170, 319-42.

Wittschieben, J., & Shuman, S. (1997) Mechanism of DNA transesterification by vaccinia topoisomerase: catalytic contributions of essential residues Arg-130, Gly-132, Tyr-136, and Lys-167. Nucleic Acids Res. 25, 3001-3008.

Yagil, E., Dorgai, L. & Weisberg, R. A. (1995). Identifying determinants of recombination specificity: construction and characterization of chimeric bacteriophage integrases. J Mol Biol 252, 163-77.

Yang, W. & Mizuuchi, K. (1997). Site-specific recombination in plane view. Structure 5(11), 1401-6.

The above examples are included for illustrative purposes only and is not intended to limit the scope of the invention. Since modifications will be apparent to those of skill in this art, it is intended that this invention be limited only by the scope of the appended claims.

What is claimed is:

1. A method of identifying a modulator of cell growth, which method comprises:
a) assessing activity of a site-specific DNA recombinase or a type I DNA topoisomerase in the presence of a test substance;
b) assessing activity of said site-specific DNA recombinase or said type I DNA topoisomerase in the absence of said test substance; and
c) comparing said activities assessed in steps a) and b);
whereby a difference in said activity assessed in step a) and said activity assessed in step b) indicates that said test substance modulates cell growth.

2. The method of claim 1, wherein the activity assessed in step a) is more than the activity assessed in step b), thereby indicating that said test substance enhances cell growth.

3. The method of claim 1, wherein the activity assessed in step a) is less than the activity assessed in step b), thereby indicating that said test substance inhibits cell growth.

4. The method of claim 1, wherein the modulator is characterized by its ability to affect overall efficiency or equilibrium of an intermediate of the DNA recombination mediated by the site-specific DNA recombinase or the type I DNA topoisomerase.

5. The method of claim 1, wherein the site-specific DNA recombinase is a tyrosine recombinase.

6. The method of claim 5, wherein the tyrosine recombinase is an eukaryotic or a prokaryotic tyrosine recombinase.

7. The method of claim 6, wherein the prokaryotic tyrosine recombinase is a bacterial tyrosine recombinase.

8. The method of claim 7, wherein the bacterial tyrosine recombinase is an eubacterial or archaebacterial tyrosine recombinase.

9. The method of claim 7, wherein the bacterial tyrosine recombinase is a gram positive or gram negative bacterial tyrosine recombinase.

10. The method of claim 7, wherein the bacterial tyrosine recombinase is derived from an enteric pathogenic bacterium.

11. The method of claim 7, wherein the bacterial tyrosine recombinase is derived from a bacterium selected from the group consisting of a SALMONELLA, a SHIGELLA, a STAPHYLOCOCCUS, a STREPTOCOCCUS and a BACILLUS species.

12. The method of claim 7, wherein the bacterial tyrosine recombinase is an E. coli. tyrosine recombinase.

13. The method of claim 7, wherein the bacterial tyrosine recombinase is a XerC, a XerD or a homolog thereof.

14. The method of claim 5, wherein the tyrosine recombinase is phage λ integrase (Int).

15. The method of claim 5, wherein the type I DNA topoisomerase is a type IA or type IB DNA topoisomerase.

16. The method of claim 15, wherein the type IA DNA topoisomerase is E. coli topoisomerase I (TopA).

17. The method of claim 15, wherein the type IB DNA topoisomerase is vaccinia virus topoisomerase.

18. The method of claim 1, wherein a tyrosine recombinase is screened against in order to identify a cell growth inhibitor and the tyrosine recombinase activity to be inhibited is selected from the group consisting of DNA strand cleavage activity, DNA strand religation activity and Holliday junction intermediate resolution activity.

19. The method of claim 18, wherein the tyrosine recombinase activity to be inhibited is the Holliday junction intermediate resolution activity.

20. The method of claim 19, wherein the Holliday junction intermediate resolution activity is assayed by conducting a tyrosine recombinase mediated recombination between two different-sized DNA duplexes, only one of said DNA duplexes is detectably labeled and successful recombination results in a detectably labeled DNA duplex with a size that is distinct from each of the original DNA duplexes, and assessing presence or amount of the Holliday junction intermediate which is resistant to protease digestion and migrates electrophoretically slower than said original DNA duplexes, said resulting recombinant DNA duplex and any covalent protein-DNA complex, whereby a test substance that increases the presence or amount of said Holliday junction intermediate indicates that said test substance inhibits the Holliday junction intermediate resolution activity of the tyrosine recombinase.

21. The method of claim 19, wherein the Holliday junction intermediate resolution activity is assayed by conducting a tyrosine recombinase mediated recombination between a DNA duplex that is capable of attaching to a solid surface and a DNA duplex that is detectably labeled, and assessing presence or amount of the Holliday junction intermediate which is both attached to said solid surface and is detectably labeled, whereby a test substance that increases the presence or amount of said Holliday junction intermediate indicates that said test substance inhibits the Holliday junction intermediate resolution activity of the tyrosine recombinase.

22. The method of claim 19, wherein the Holliday junction intermediate resolution activity is assayed by conducting a tyrosine recombinase mediated recombination between a DNA duplex with a first label and a DNA duplex with a second label, and assessing presence or amount of the Holliday junction intermediate which gives a detectable signal resulted from proximity of said first and second label in the Holliday junction and said detectable signal is detectably distinct from the signal of said first and second label, whereby a test substance that increases the presence or amount of said Holliday junction intermediate indicates that said test substance inhibits the Holliday junction intermediate resolution activity of the tyrosine recombinase.

23. The method of claim 22, wherein the first label and the second label are components of a FRET detection system.

24. The method of claim 14, wherein an Int inhibitor is identified by its ability of decreasing overall efficiency of the Int-mediated recombination or its ability of accumulating or stabilizing a Holliday junction or synaptic intermediate.

25. The method of claim 1, wherein the test substance is a peptide.

26. The method of claim 25, wherein the peptide has at least four amino acid residues.

27. The method of claim 1, wherein a plurality of test substances is assayed simultaneously.

28. A cell growth modulator identified according to the method of claim 1.

29. An isolated peptide for inhibiting a tyrosine recombinase, which peptide has the following formula:
(Xaa1-Xaa2-Xaa3-Xaa4)n
wherein Xaa1 is an aromatic or a basic amino acid residue, Xaa2 is Ser, Cys, an aromatic or a basic amino acid residue, Xaa3 is any amino acid residue, Xaa4 is Ser, Cys, an aromatic or a basic amino acid residue, wherein each of Xaa1, Xaa2, Xaa3, or Xaa4 can be a D or L amino acid residue and wherein n is an integer ranging from 1 to 10.

30. The isolated peptide of claim 29, wherein Xaa1 is Trp, Arg or Tyr; Xaa2 is Trp, Lys, Arg or Cys; Xaa3 is Ala, His, Val, Arg, Trp, Tyr or Cys; and Xaa4 is Trp, Cys, Tyr, Arg or Phe.

31. The isolated peptide of claim 29, which is selected from the group consisting of:
1) Trp-Lys-Ala-Tyr;
2) Trp-Lys-His-Tyr;
3) Trp-Lys-Val-Tyr;
4) Trp-Arg-Arg-Trp;
5) Trp-Arg-Trp-Tyr;
6) Trp-Arg-Arg-Cys;
7) Trp-Arg-Tyr-Arg;
8) Arg-Cys-Trp-Trp;
9) Arg-Cys-Cys-Tyr; and
10) Tyr-Trp-Cys-Tyr.

32. The isolated peptide of claim 29, further comprising a Met as the first N-terminal amino acid residue.

33. An isolated peptide for inhibiting a tyrosine recombinase, which peptide has the following formula:
(Xaa1-Xaa2-Xaa3-Xaa4-Xaa5)n
wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, wherein each of Xaa1, Xaa2, Xaa3, Xaa4 or Xaa5 can be a D or L amino acid residue and wherein n is an integer ranging from 1 to 10.

34. The isolated peptide of claim 33, wherein Xaa1 is Trp, Arg or Tyr; Xaa2 is Trp, Lys, Arg or Cys; Xaa3 is Ala, His, Val, Trp, Arg, Cys or Tyr; Xaa4 is Trp, Cys, Tyr Phe or Arg; and Xaa5 is Gln, Pro, Cys, Arg or Trp.

35. The isolated peptide of claim 33, which is selected from the group consisting of:
1) Trp-Lys-Ala-Tyr-Gln;
2) Trp-Lys-His-Tyr-Pro;
3) Trp-Lys-His-Tyr-Gln;
4) Trp-Lys-Val-Tyr-Pro;
5) Trp-Lys-Val-Tyr-Gln;
6) Trp-Lys-Ala-Tyr-Pro;
7) Trp-Arg-Arg-Trp-Cys;
8) Trp-Arg-Trp-Tyr-Cys;
9) Trp-Arg-Arg-Cys-Arg;
10) Trp-Arg-Tyr-Arg-Cys;
11) Arg-Cys-Trp-Trp-Trp;
12) Arg-Cys-Cys-Tyr-Trp;
13) Tyr-Trp-Cys-Tyr-Trp; and
14) Trp-Lys-His-Phe-Gln.

36. The isolated peptide of claim 33, further comprising a Met as the first N-terminal amino acid residue.

37. An isolated peptide for inhibiting a tyrosine recombinase, which peptide has the following formula:
(Xaa1-Xaa2-Xaa3-Xaa4-Xaa5-Xaa6)n
wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, Xaa6 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

38. The isolated peptide of claim 37, wherein Xaa1 is Trp, Arg or Tyr; Xaa2 is Trp, Lys, Arg or Cys; Xaa3 is Ala, His, Val, Trp, Arg, Cys or Tyr; Xaa4 is Trp, Cys, Tyr Phe or Arg; Xaa5 is Gln, Pro, Cys, Arg or Trp; and Xaa6 is Tyr, Arg, Phe or Trp.

39. The isolated peptide of claim 35, which is selected from the group consisting of:
1) Trp-Lys-Ala-Tyr-Gln-Tyr;
2) Trp-Lys-His-Tyr-Pro-Tyr;
3) Trp-Lys-His-Tyr-Gln-Tyr;
4) Trp-Lys-Val-Tyr-Pro-Tyr;
5) Trp-Lys-Val-Tyr-Gln-Tyr;
6) Trp-Lys-Ala-Tyr-Pro-Tyr;
7) Trp-Arg-Arg-Trp-Cys-Arg;
8) Trp-Arg-Trp-Tyr-Cys-Arg;
9) Trp-Arg-Arg-Cys-Arg-Trp;
10) Trp-Arg-Tyr-Arg-Cys-Arg;
11) Arg-Cys-Trp-Trp-Trp-Trp;
12) Arg-Cys-Cys-Tyr-Trp-Trp;
13) Tyr-Trp-Cys-Tyr-Trp-Trp;
14) Trp-Lys-His-Phe-Gln-Tyr; and
15) Trp-Lys-His-Tyr-Gln-Phe.

40. The isolated peptide of claim 37, further comprising a Met as the first N-terminal amino acid residue.

41. An isolated peptide for inhibiting a tyrosine recombinase, which peptide is selected from the group consisting of:
1) Met-Trp-Lys-His-Tyr-Gln-Tyr;
2) Trp-Lys-His-Tyr-Gln-Tyr-Lys-Trp-Lys-His-Tyr-Gln-Tyr; and
3) Trp-Lys-His-Tyr-Gln-Tyr wherein each of the six amino acid residues is a D amino acid residue.

42. An isolated peptide for inhibiting a tyrosine recombinase or a type I DNA topoisomerase, which peptide has the following formula:
(Xaa1-Xaa2-Xaa3-Xaa4)n
wherein each of Xaa1 and Xaa2 is an aromatic amino acid residue, Xaa3 is Ser, Cys or an aromatic amino acid residue, Xaa4 is an aromatic or a basic amino acid residue, wherein each of Xaa1, Xaa2, Xaa3, or Xaa4 can be a D or L amino acid residue and wherein n is an integer ranging from 1 to 10.

43. The isolated peptide of claim 42, wherein Xaa1 is Trp; Xaa2 is Trp; Xaa3 is Trp or Cys; and Xaa4 is Trp or Arg.

44. The isolated peptide of claim 42, which is selected from the group consisting of:
1) Trp-Trp-Trp-Trp;
2) Trp-Trp-Trp-Arg;
3) Trp-Trp-Cys-Trp; and
4) Trp-Trp-Cys-Arg.

45. The isolated peptide of claim 42, further comprising a Met as the first N-terminal amino acid residue.

46. An isolated peptide for inhibiting a tyrosine recombinase or a type I DNA topoisomerase, which peptide has the following formula:
(Xaa1-Xaa2-Xaa3-Xaa4-Xaa5)n
wherein Xaa1 is a basic amino acid residue, each of Xaa2 and Xaa3 is an aromatic amino acid residue, Xaa4 is Ser, Cys or an aromatic amino acid residue, Xaa5 is an aromatic or a basic amino acid residue, wherein each of Xaa1, Xaa2, Xaa3, Xaa4 or Xaa5 can be a D or L amino acid residue and wherein n is an integer ranging from 1 to 10.

47. The isolated peptide of claim 46, wherein Xaa1 is Lys or Arg; Xaa2 is Trp; Xaa3 is Trp; Xaa4 is Trp or Cys; and Xaa5 is Trp or Arg.

48. The isolated peptide of claim 46, which is selected from the group consisting of:
1) Lys-Trp-Trp-Trp-Trp;
2) Lys-Trp-Trp-Trp-Arg;
3) Lys-Trp-Trp-Cys-Trp; and
4) Lys-Trp-Trp-Cys-Arg.

49. The isolated peptide of claim 46, further comprising a Met as the first N-terminal amino acid residue.

50. An isolated peptide for inhibiting a tyrosine recombinase or a type I DNA topoisomerase, which hexapeptide has the following formula:
(Xaa1-Xaa2-Xaa3-Xaa4-Xaa5-Xaa6)n
wherein Xaa1 is a basic amino acid residue, each of Xaa2 and Xaa3 is an aromatic amino acid residue, each of Xaa4 and Xaa6 is Ser, Cys or an aromatic amino acid residue, Xaa5 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

51. The isolated peptide of claim 50, wherein Xaa1 is Lys; Xaa2 is Trp; Xaa3 is Trp; Xaa4 is Trp or Cys; Xaa5 is Trp or Arg; and Xaa6 is Trp or Cys.

52. The isolated peptide of claim 50, which is selected from the group consisting of:
1) Lys-Trp-Trp-Trp-Trp-Trp;
2) Lys-Trp-Trp-Trp-Arg-Trp;
3) Lys-Trp-Trp-Trp-Trp-Cys;
4) Lys-Trp-Trp-Cys-Trp-Trp;
5) Lys-Trp-Trp-Cys-Arg-Trp; and
6) Lys-Trp-Trp-Cys-Trp-Cys.

53. The isolated peptide of claim 50, further comprising a Met as the first N-terminal amino acid residue.

54. An isolated peptide for inhibiting a tyrosine recombinase or a type I DNA topoisomerase, which peptide is selected from the group consisting of:
1) Met-Lys-Trp-Trp-Cys-Arg-Trp;
2) Arg-Cys-Trp-Trp-Trp-Trp; and
3) Trp-Cys-Trp-Trp-Trp-Trp.

55. A method for inhibiting cell growth in a subject, which method comprises administering to a subject, to which such inhibition is desirable, an effective amount of an inhibitor of a site-specific DNA recombinase or a type I DNA topoisomerase, whereby cell growth is inhibited.

56. The method of claim 55, wherein the subject is a mammal.

57. The method of claim 56, wherein the mammal is a human.

58. The method of claim 55, further comprising administering a pharmaceutically acceptable carrier or excipient.

59. The method of claim 55, wherein the inhibitor inhibits a tyrosine recombinase or a type I DNA topoisomerase.

60. The method of claim 55, wherein the subject has or is suspected of having tumor or cancer.

61. The method of claim 60, further comprising administering an effective amount of an anti-tumor or anti-cancer agent or treatment.

62. The method of claim 55, wherein the subject is or is suspected of being infected by a bacterium and the inhibitor inhibits Holliday junction intermediate resolution activity of a tyrosine recombinase.

63. The method of claim 62, further comprising administering an effective amount of an antibiotic or an anti-bacterium treatment.

64. The method of claim 55, wherein the inhibitor of a tyrosine recombinase is selected from the group consisting of:
1) a peptide having the following formula:
(Xaa1-Xaa2-Xaa3-Xaa4)n
wherein Xaa1 is an aromatic or a basic amino acid residue, Xaa2 is Ser, Cys, an aromatic or a basic amino acid residue, Xaa3 is any amino acid residue, Xaa4 is Ser, Cys, an aromatic or a basic amino acid residue, wherein each of Xaa1, Xaa2, Xaa3, or Xaa4 can be a D or L amino acid residue and wherein n is an integer ranging from 1 to 10;
2) a peptide having the following formula:
(Xaa1-Xaa2-Xaa3-Xaa4-Xaa5)n
wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, wherein each of Xaa1, Xaa2, Xaa3, Xaa4 or Xaa5 can be a D or L amino acid residue and wherein n is an integer ranging from 1 to 10; and
3) a peptide having the following formula:
(Xaa1-Xaa2-Xaa3-Xaa4-Xaa5-Xaa6)n
wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, Xaa6 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

65. The method of claim 55, wherein the inhibitor of a site-specific DNA recombinase is selected from the group consisting of:
1) a peptide having the following formula:
(Xaa1-Xaa2-Xaa3-Xaa4)n
wherein each of Xaa1 and Xaa2 is an aromatic amino acid residue, Xaa3 is Ser, Cys or an aromatic amino acid residue, Xaa4 is an aromatic or a basic amino acid residue, wherein each of Xaa1, Xaa2, Xaa3, or Xaa4 can be a D or L amino acid residue and wherein n is an integer ranging from 1 to 10.
2) a peptide having the following formula:
(Xaa1-Xaa2-Xaa3-Xaa4-Xaa5)n
wherein Xaa1 is a basic amino acid residue, each of Xaa2 and Xaa3 is an aromatic amino acid residue, Xaa4 is Ser, Cys or an aromatic amino acid residue, Xaa5 is an aromatic or a basic amino acid residue, wherein each of Xaa1, Xaa2, Xaa3, Xaa4 or Xaa5 can be a D or L amino acid residue and wherein n is an integer ranging from 1 to 10; and
3) a peptide having the following formula:
(Xaa1-Xaa2-Xaa3-Xaa4-Xaa5-Xaa6)n
wherein Xaa1 is a basic amino acid residue, each of Xaa2 and Xaa3 is an aromatic amino acid residue, each of Xaa4 and Xaa6 is Ser, Cys or an aromatic amino acid residue, Xaa5 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

66. An isolated and labeled peptide selected from the group consisting of:
1) a peptide having the following formula:
(Xaa1-Xaa2-Xaa3-Xaa4)n
wherein Xaa1 is an aromatic or a basic amino acid residue, Xaa2 is Ser, Cys, an aromatic or a basic amino acid residue, Xaa3 is any amino acid residue, Xaa4 is Ser, Cys, an aromatic or a basic amino acid residue, wherein each of Xaa1, Xaa2, Xaa3, or Xaa4 can be a D or L amino acid residue and wherein n is an integer ranging from 1 to 10;
2) a peptide having the following formula:
(Xaa1-Xaa2-Xaa3-Xaa4-Xaa5)n
wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, wherein each of Xaa1, Xaa2, Xaa3, Xaa4 or Xaa5 can be a D or L amino acid residue and wherein n is an integer ranging from 1 to 10; and
3) a peptide having the following formula:
(Xaa1-Xaa2-Xaa3-Xaa4-Xaa5-Xaa6)n
wherein Xaa1 is an aromatic or a basic amino acid residue, Xaa2 is Ser, Cys, an aromatic or a basic amino acid residue, each of Xaa3 or Xaa5 is any amino acid residue, Xaa4 is Ser, Cys, an aromatic or a basic amino acid residue, Xaa6 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

67. The isolated and labeled peptide of claim 66, wherein the label is selected from the group consisting of a chemical, an enzymatic, an radioactive, a fluorescent and a luminescent label.

68. The isolated and labeled peptide of claim 66, which is biotinylated or fluorescently labeled at a Cys or Lys residue.

69. An isolated and labeled peptide selected from the group consisting of:
1) a peptide having the following formula:
(Xaa1-Xaa2-Xaa3-Xaa4)n
wherein each of Xaa1 and Xaa2 is an aromatic amino acid residue, Xaa3 is Ser, Cys or an aromatic amino acid residue, Xaa4 is an aromatic or a basic amino acid residue, wherein each of Xaa1, Xaa2, Xaa3, or Xaa4 can be a D or L amino acid residue and wherein n is an integer ranging from 1 to 10.
2) a peptide having the following formula:
(Xaa1-Xaa2-Xaa3-Xaa4-Xaa5)n
wherein Xaa1 is a basic amino acid residue, each of Xaa2 and Xaa3 is an aromatic amino acid residue, Xaa4 is Ser, Cys or an aromatic amino acid residue, Xaa5 is an aromatic or a basic amino acid residue, wherein each of Xaa1, Xaa2, Xaa3, Xaa4 or Xaa5 can be a D or L amino acid residue and wherein n is an integer ranging from 1 to 10; and
3) a peptide having the following formula:
(Xaa1-Xaa2-Xaa3-Xaa4-Xaa5-Xaa6)n
wherein Xaa1 is a basic amino acid residue, each of Xaa2 and Xaa3 is an aromatic amino acid residue, each of Xaa4 and Xaa6 is Ser, Cys or an aromatic amino acid residue, Xaa5 is an aromatic or a basic amino acid residue, wherein each of Xaa1, Xaa2, Xaa3, Xaa4, Xaa5 or Xaa6 can be a D or L amino acid residue and wherein n is an integer ranging from 1 to 10.

70. The isolated and labeled peptide of claim 69, wherein the label is selected from the group consisting of a chemical, an enzymatic, an radioactive, a fluorescent and a luminescent label.

71. The isolated and labeled peptide of claim 69, which is biotinylated or fluorescently labeled at a Cys or Lys residue.
